SEQUENCE LISTING (38 sequences)

SEQ ID NO: 1 - protein, 46 aa, synthetic construct
NGLLIPQFLVASGGGGSRSIVTFSLRQNRGGGGSNGLLIPQFLVAS

SEQ ID NO: 2 - protein, 49 aa, synthetic construct
NGLLIPQFLVASGGGGSRALQALRALQALEALGGGGSNGLLIPQFLVAS

SEQ ID NO: 3 - protein, 49 aa, synthetic construct
NGLLIPQFLVASGGGGSRALRALQALEALEALGGGGSNGLLIPQFLVAS

SEQ ID NO: 4 - protein, 12 aa, synthetic construct
RSIVTFSLRQNR

SEQ ID NO: 5 - protein, 15 aa, synthetic construct
RALQALRALQALEAL

SEQ ID NO: 6 - protein, 15 aa, synthetic construct
RALRALQALEALEAL

SEQ ID NO: 7 - protein, 25 aa, synthetic construct
NGLLIPQFLVASGGGGSMSDYQMDM

SEQ ID NO: 8 - protein, 28 aa, synthetic construct
RALRALQALEALEALGGGGSMSDYQMDM

SEQ ID NO: 9 - protein, 28 aa, synthetic construct
RALQALRALQALEALGGGGSMSDYQMDM

SEQ ID NO: 10 - protein, 27 aa, synthetic construct
NGLLIPQFLVASGGGGSMDMQGRYMDR

SEQ ID NO: 11 - protein, 30 aa, synthetic construct
RALRALQALEALEALGGGGSMDMQGRYMDR

SEQ ID NO: 12 - protein, 30 aa, synthetic construct
RALQALRALQALEALGGGGSMDMQGRYMDR

SEQ ID NO: 13 - protein, 5 aa, synthetic construct
GGGGS

SEQ ID NO: 14 - protein, 10 aa, synthetic construct
GGGGSGGGGS

SEQ ID NO: 15 - protein, 15 aa, synthetic construct
GGGGSGGGGSGGGGS

SEQ ID NO: 16 - protein, 22 aa, synthetic construct
GGGGSGGGGSGGGGSGGGGGGS

SEQ ID NO: 17 - protein, 8 aa, synthetic construct
GGGGGGGG

SEQ ID NO: 18 - protein, 6 aa, synthetic construct
GGGGGG

SEQ ID NO: 19 - protein, 5 aa, synthetic construct
EAAAK

SEQ ID NO: 20 - protein, 10 aa, synthetic construct
EAAAKEAAAK

SEQ ID NO: 21 - protein, 15 aa, synthetic construct
EAAAKEAAAKEAAAK

SEQ ID NO: 22 - protein, 46 aa, synthetic construct
AEAAAKEAAAKEAAAKEAAAKALEAAAAAKEAAAKEAAAKEAAAKA

SEQ ID NO: 23 - protein, 12 aa, synthetic construct
AEAAAKEAAAKA

SEQ ID NO: 24 - protein, 5 aa, synthetic construct
PAPAP

SEQ ID NO: 25 - protein, 4 aa, synthetic construct
SGGG

SEQ ID NO: 26 - protein, 4 aa, synthetic construct
GGGS

SEQ ID NO: 27 - protein, 64 aa, synthetic construct
MYMDMKDVMMKYDMAMMEMKMDSGGGRSIVTFSLRQNRGGGSMYMDMKDVMMKYDMAMMEMKMD

SEQ ID NO: 28 - protein, 56 aa, synthetic construct
MYMDMKDVMMKYDMAMMEMKMDRSIVTFSLRQNRMYMDMKDVMMKYDMAMMEMKMD

SEQ ID NO: 29 - protein, 28 aa, synthetic construct
MKMYMKMDRSIVTFSLRQNRMKMYMKMD

SEQ ID NO: 30 - protein, 26 aa, synthetic construct
MMYMMYMRSIVTFSLRQNRMMYMMYM

SEQ ID NO: 31 - skipped

SEQ ID NO: 32 - protein, 64 aa, synthetic construct
KYGDMKDVKMKYDKACSEMKYDSGGGRSIVTFSLRQNRGGGSKYGDMKDVKMKYDKACSEMKYD

SEQ ID NO: 33 - protein, 56 aa, synthetic construct
KYGDMKDVKMKYDKACSEMKYDRSIVTFSLRQNRKYGDMKDVKMKYDKACSEMKYD

SEQ ID NO: 34 - protein, 22 aa, synthetic construct
MYMDMKDVMMKYDMAMMEMKMD

SEQ ID NO: 35 - protein, 8 aa, synthetic construct
MKMYMKMD

SEQ ID NO: 36 - protein, 7 aa, synthetic construct
MMYMMYM

SEQ ID NO: 37 - protein, 12 aa, synthetic construct
NGLLIPQFLVAS

SEQ ID NO: 38 - protein, 22 aa, synthetic construct
KYGDMKDVKMKYDKACSEMKYD

(19) United States
(12) Patent Application Publication
(10) Pub. No.: US 2025/0333666 A1
(43) Pub. Date: Oct. 30, 2025

(54) LIQUID DETERGENT AND UNIT DOSE DETERGENT COMPOSITIONS AND METHODS OF USING THE SAME

(71) Applicant: Henkel AG & Co. KGaA, Duesseldorf (DE)

(72) Inventors: Daniel Thomas PIORKOWSKI, Fairfield, CT (US); Kyle Seamus Joseph O‘Connor, Stamford, CT (US); Fabian Falkenberg, Neuss (DE); Christian Degering, Erkrath (DE)

(73) Assignee: Henkel AG & Co. KGaA, Duesseldorf (DE)

(21) Appl. No.: 19/189,482

(22) Filed: Apr. 25, 2025

Related U.S. Application Data
(60) Provisional application No. 63/639,023, filed on Apr. 26, 2024.

(51) Int. Cl. C11D 3/386; C07K 7/06; C07K 7/08; C07K 14/00; C11D 1/14; C11D 1/72; C11D 1/83; C11D 3/43; C11D 17/04; C12N 9/26; C12N 9/48

(52) U.S. Cl. CPC C11D 3/38618; C07K 7/06; C07K 7/08; C07K 14/001; C11D 1/146; C11D 1/72; C11D 1/83; C11D 3/43; C11D 17/043; C12N 9/2411; C12N 9/48; C07K 2319/00; C11D 2111/12; C12Y 302/01

(57) ABSTRACT

Disclosed herein are peptides, unit dose or liquid detergent compositions comprising peptides, and methods of making and using the same. The unit dose or liquid detergent compositions include at least one peptide, at least one anionic surfactant, at least one non-ionic surfactant, and water. The at least one peptide contains binding domains having a binding affinity to a textile; and two or more amino acids susceptible to oxidation.

### CROSS-REFERENCE TO A RELATED APPLICATION

This patent application claims the benefit of U.S. Provisional Patent Application No. 63/639,023, filed Apr. 26, 2024, the disclosure of which is incorporated by reference for all purposes.

### REFERENCE TO SEQUENCE LISTING SUBMITTED ELECTRONICALLY

The content of the electronically submitted sequence listing in ASCII text file (2818_8470001_SequenceListing_ST 26.xml; Size: 33,881 bytes; and Date of Creation: Apr. 24, 2025) filed with the application is incorporated herein by reference in its entirety.

### FIELD OF THE INVENTION

This disclosure generally relates to unit dose or liquid detergent packs and liquid detergent compositions and methods of using the same. More specifically, this disclosure generally relates to unit dose or liquid detergent packs and liquid detergent compositions containing peptides.

### BACKGROUND

In unit dose or liquid detergent packs and liquid detergent compositions, amino acids such as lysine can provide a benefit with scavenging chlorine. This is of importance since wash water sourced from the city contains chlorine as a sanitization agent against microbes (so it is potable/drinkable). Chlorine is known to deteriorate fabrics' dyes over time, particularly red, blue or black dyes. This dye deterioration will be seen in 3 to 30 washes, if not controlled. In addition to fabric dye damage, chlorine in the water can negatively impact an enzyme's performance for cleaning soiled laundry. Lysine has been shown to remove chlorine from the wash, as quickly as seconds. There is a need for a detergent composition that can effectively clean textiles while scavenging chlorine to protect fabric dyes and enzyme activity, in both of the wash and rinse cycles.

### SUMMARY OF THE INVENTION

The present disclosure relates to a peptide comprising: (i) one or more textile binding domains, wherein the one or more textile binding domains comprise an amino acid sequence having at least 70%, at least 75%, at least 80%, at least 85%, at least 90%, at least 95%, at least 97%, at least 99%, or 100% identity to SEQ ID NO: 4 and (ii) one or more domains susceptible to oxidation, wherein the one or more domains susceptible to oxidation comprise an amino acid sequence having at least 70%, at least 75%, at least 80%, at least 85%, at least 90%, at least 95%, at least 97%, at least 99%, or 100% identity to any of SEQ ID NOs: 34-38.

In some aspects, the present disclosure also relates to a peptide comprising (i) one or more textile binding domains; and (ii) one or more domains susceptible to oxidation, wherein the domains susceptible to oxidation are selected from: (1) a domain comprising about 45% of the amino acids of the one or more domains susceptible to oxidation are M and about 55% of the amino acids are selected from Y, D, K, V, A, and E; (2) a domain comprising about 50% of the amino acids of the one or more domains susceptible to oxidation are M and about 50% of the amino acids are selected from K, Y, and D; (3) a domain comprising about 70% of the amino acids of the one or more domains susceptible to oxidation are M and about 30% of the amino acids are Y; (4) a domain comprising about 25% of the amino acids of the one or more domains susceptible to oxidation are L and about 75% of the amino acids are N, G, I, P, Q, F, V, A, and S; and (5) a domain comprising about 40% of the amino acids of the one or more domains susceptible to oxidation are selected from M and K and about 60% of the amino acids are selected from Y, G, D, V, A, C, S, and E.

In some aspects, the present disclosure relates to a peptide comprising an amino acid sequence having at least 70%, at least 75%, at least 80%, at least 85%, at least 90%, at least 95%, at least 97%, at least 99%, or 100% identity to any of SEQ ID NOs: 1-3, 5-12, 27-30, and 32-33.

In some aspects, the peptide comprises one textile binding domain and two domains susceptible to oxidation.

In some aspects, the peptide further comprises one or more linking domains.

In some aspects, the peptide comprises one textile binding domain, two domains susceptible to oxidation, and two linking domains.

In some aspects, the peptide comprises an amino acid sequence having at least 70%, at least 75%, at least 80%, at least 85%, at least 90%, at least 95%, at least 97%, at least 99%, or 100% identity to any of SEQ ID NOs: 1-3, 5-12, 27-30, and 32-33.

In some aspects, the one or more domains susceptible to oxidation comprise an amino acid sequence having at least 70%, at least 75%, at least 80%, at least 85%, at least 90%, at least 95%, at least 97%, at least 99%, or 100% identity to any of SEQ ID NOs: 34-38.

In some aspects, the one or more textile binding domain comprises an amino acid sequence having at least 70%, at least 75%, at least 80%, at least 85%, at least 90%, at least 95%, at least 97%, at least 99%, or 100% identity to SEQ ID NO: 4.

In some aspects, the disclosure relates to a detergent composition comprising a peptide as described herein.

In some aspects, the detergent composition further comprises (a) about 1 to about 30 wt % of a non-ionic surfactant; (b) about 1 to about 35 wt % of an anionic surfactant; (c) about 15 to about 65 wt % of a non-aqueous solvent or non-aqueous co-solvent mixture; and (d) water, wherein the peptide is present in an amount ranging from about 0.01 to about 5 wt % of the detergent composition.

In some aspects, the non-ionic surfactant comprises an alkoxylated alcohol.

In some aspects, the anionic surfactant comprises a linear alkyl sulfonate, a linear alkylbenzene sulfonate, a sodium lauryl sulfate, a sodium lauryl ether sulfate, and any combination thereof.

In some aspects, the non-aqueous solvent or non-aqueous co-solvent mixture comprises glycerin, propylene glycol, polyethylene glycol, ethanol, and any combination thereof.

In some aspects, detergent composition further comprises at least one ingredient selected from the group consisting of: a fatty acid, a strong base, one or more enzymes, a performance polymer, an alkanolamine, a fragrance, a dye, and any combination thereof.

In some aspects, the detergent composition comprises one or more enzymes.

In some aspects, the detergent composition has a pH of about 7 to about 9.

In some aspects, the detergent composition further comprises a pouch encapsulating the detergent composition, wherein the pouch is made of a water-soluble material.

In some aspects, the present disclosure relates to a method of cleaning textiles, the method comprising combining the detergent composition as described herein with water to form a diluted detergent composition having from about 1 to about 2 grams of the detergent composition per liter of water; and cleaning the textiles using the diluted detergent composition.

In some aspects, the textiles are made of natural materials, synthetic materials or blends thereof.

### DETAILED DESCRIPTION

The following description of the embodiments is merely exemplary in nature and is in no way intended to limit the subject matter of the present disclosure, their application, or uses.

### Definitions

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all aspects by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, alternatively ±1 percent, alternatively ±0.5 percent, and alternatively ±0.1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the present invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The term “conservative amino acid substitution” means the substitution of an amino acid residue for another amino acid residue, whereby this exchange does not result in a change in polarity or charge at the position of the exchanged amino acid, e.g. the exchange of a non-polar amino acid residue for another non-polar amino acid residue. Conservative amino acid substitutions within the scope of the invention include, for example: G=A=S, I=V=L=M, D=E, N=Q, K=R, Y=F, S=T, G=A=|=V=L=M=Y=F=W=P=S=T. However, it may be preferable that such exchanges do not have glycine or tyrosine as the target amino acid, or e.g. no amino acid that has a low alpha-helix-forming potential.

As used herein, the term “textile” means fibers, yarns, woven or unwoven fabrics, cloths and so on. The textiles may be made of natural materials, synthetic materials or blends thereof. Example natural materials include, but are not limited to, cotton, wool, cashmere, mohair, alpaca hair, silk, linen, jute, and cellulosic fibers. Example synthetic materials synthetic materials include, but are not limited to polyester (PES), polyethylene (PE), polypropylene (PP), polyurethane (PU), polystyrene (PS), polyvinyl chloride (PVC), polycarbonate (PC), polyamide (PA), polyphenylene ether, polyphenylene sulfide, polyoxymethylene (POM), polymethyl methacrylate (PMA), polyethylene terephthalate (PET), polybutylene terepthalathe (PBT), polytetrafluoroethylene (PTFE), polyhydroxyalkanoate (PHA), polyhydroxybutyrate (PHB), polyimide (PI), polylactide (PLA), polyvinylidene fluoride (PV DF), Polyetherketone (PEK), and/or copolymers or blends thereof. Blended textiles can be made of at least one natural material and at least one synthetic material. Blended textiles can have a natural material to synthetic material weight:weight ratio ranging from, for example 5:95 to 95:5, alternatively from 10:90 to 90:10, alternatively from 15:85 to 85:15, alternatively from 20:80 to 80:20, alternatively from 25:75 to 75:25, alternatively from 30:70 to 70:30, alternatively from 35:65 to 65:35, alternatively from 40:60 to 60:40, and alternatively from 45:55 to 55:45.

As used herein, “water soluble” means at least 2 grams of the solute (the film in one example) will dissolve in 5 liters of water, for a solubility of at least 0.4 grams per liter at a temperature of 25 degrees Celsius (° C.) unless otherwise specified.

Various aspects of the present disclosure are directed towards unit dose or liquid detergent products and methods of using the same.

In accordance with various aspects of the disclosure, certain unit dose or liquid detergent products comprise, consist essentially of, or consist of a pouch comprising at least one chamber, the pouch made of a water-soluble material, and a detergent composition encapsulated with within the at least one chamber, the detergent composition. The detergent composition can comprise, consist essentially of, or consist of a peptide, a nonionic surfactant, an anionic surfactant, and water. In accordance with various aspects of the disclosure, the inventors have discovered the use of certain peptides in laundry detergent compositions serves at least two important purposes. First, the inventors have discovered that certain peptides serve as effective chlorine scavengers when used in laundry detergent compositions. This is of importance since both wash and rinse water (especially water from municipal sources) contain chlorine as a sanitization agent against microbes. Chlorine deteriorated dyes in textiles over time, which may be observed (by, for example, reduced color saturation or intensity exhibited by the textile) in 30 washes or less if not controlled. In addition to fabric dye damage, chlorine can negatively impact the cleaning performance of enzymes that may be present in laundry detergent compositions. Further the peptides can be engineered to adhere to the textile, so they can carry over to the rinse water and scavenge chlorine in the rinse. This will provide a color care benefit, since scent booster/detergents added to the wash will only scavenge chlorine in the wash, leaving textiles susceptible to chlorine in the rinse. Products such as fabric softener (containing ester quats) will scavenge chlorine in the rinse (since they are added to the rinse), but not all consumers use fabric softeners. The use of a specialized peptide will provide the consumer with the benefit of scavenging chlorine in both the wash and rinse, providing better color care to their textiles. As such, the use of peptides as a chlorine scavenger and textile binder can help enhance enzyme cleaning performance and thus the overall cleaning efficiency of the laundry detergent composition through its use.

A unit dose pack is formed by encapsulating a detergent composition within a container, where the container is made in part or completely of a film. In some aspects, the film forms a portion, such as one half, or more of the container. In some aspects, the container may also include dyes, print, or other components in or on a surface of the container. The film is water soluble such that the film will completely dissolve when exposed to water, such as in a washing machine typically used for laundry. When the film dissolves, the container ruptures and the contents are released. Suitable films for packaging are rapidly and completely soluble in water at temperatures of about 5° C. or greater. In some aspects, the container comprises, consists essentially of, or consists of the pouch comprising at least one chamber discussed above.

In some aspects, the film is desirably strong, flexible, shock resistant, transparent, and non-tacky during storage at both high and low temperatures and high and low humidity. In some aspects, the film is initially formed from polyvinyl acetate, and at least a portion of the acetate functional groups are hydrolyzed to produce alcohol groups. Therefore, the film includes polyvinyl alcohol (PVOH), and may include a higher concentration of PVOH than polyvinyl acetate. Such films are commercially available with various levels of hydrolysis, and thus various concentrations of PVOH. In some aspects, the film initially has about 85 to about 90 percent of the acetate groups hydrolyzed to alcohol groups, but other percentages of hydrolysis are also possible in alternate aspects. In some aspects, some of the acetate groups may further hydrolyze in use, so the final concentration of alcohol groups may be higher than the concentration at the time of packaging. In some aspects, the film may have a thickness of from about 25 to about 200 microns (μm), alternatively from about 45 to about 100 μm, or alternatively from about 65 to about 90 μm. In some aspects, the film may include alternate materials such as methyl hydroxy propyl cellulose and polyethylene oxides. Generally, the type of water-soluble material used for the film is not limiting, however the type of material used for the film should be soluble in water.

In some aspects, unit dose packs may be formed from a container having a single section (or “pouch”). In some aspects, unit dose packs may be formed from containers with two or more different sections (or “pouches”). In some aspects, when a container has two or more sections, the contents of the different sections may or may not be the same. In some aspects, with two or more sections, at least one of the sections includes the detergent composition. In some aspects, the other section may include the same or a different formulation of detergent composition, but in some aspects the other section includes a different composition, such as a fabric softening composition or other fabric treatment. In some aspects, the unit dose pack is formulated and configured for cleaning laundry, but other cleaning purposes are also possible. In some aspects, the detergent composition is positioned within the container, and the container is sealed to encapsulate and enclose the detergent composition. In some aspects, the detergent composition is in direct contact with the film of the container within the unit dose pack. In some aspects, the film of the container is sealable by heat, a combination of heat and water, ultrasonic methods, or other suitable techniques. In some aspects, a combination of sealing techniques may be used to enclose the detergent composition within the container. In some aspects, the water-soluble material is a polyvinyl alcohol. In some aspects, the water-soluble material is a polyethylene terephthalate. In some aspects, the water-soluble material is a polyethylene, a polypropylene or a polyvinyl chloride.

Various aspects of the present disclosure are directed to liquid detergent compositions contained within unit dose packs and methods of using the same. In accordance with various aspects of the disclosure, certain liquid detergent compositions comprise, consist essentially of, or consist of a peptide, an anionic surfactant, a non-ionic surfactant, and water

### Peptides

Certain aspects of the disclosure are directed to peptides comprising one or more textile binding domains and one or more domains susceptible to oxidation. In some aspects, the peptide comprises one textile binding domain and two domains susceptible to oxidation. In some aspects, the peptide comprises one or more linking domains. In some aspects, the peptide comprises one textile binding domain, two domains susceptible to oxidation, and two linking domains.

Liquid detergent compositions according to the disclosure include a peptide that has the ability to adhere to a textile and includes amino acids susceptible to oxidation in the presence of, for example, chlorine as described elsewhere herein. Peptides for use in liquid detergents according to the disclosure can range from about 5 to about 200 amino acids in length. In some aspects, the peptide can range from about 5 to about 100 amino acids in length. In some aspects, the peptide can range from about 5 to about 75 amino acids in length. In some aspects, the peptide can range from about 10 to about 60 amino acids in length. In some aspects, the peptide is between about 20 to about 70 amino acids in length. In some aspects, the peptide is between about 25 to about 65 amino acids in length. In some aspects, the peptide is between about 25 to about 30 amino acids, about 30 to about 35 amino acids, about 40 to about 45 amino acids, about 45 to about 50, about 50 to about 55, about 55 to about 60, about 60 to about 65, or about 65 to about 70 amino acids in length. In some aspects, the peptide is about 26, about 28, about 46, about 56, or about 64 amino acids in length.

In some embodiments of the present invention, the laundry detergent comprises a peptide, wherein the peptide comprises an amino acid sequence having at least 70%, at least 75%, at least 80%, at least 85%, at least 90%, at least 92%, at least 95%, at least 97%, at least 99%, or 100% identity to any one of SEQ ID NOs: 1-3, 5-12, 27-30, and 32-33. In some aspects, the peptide has the amino acid sequence of any of SEQ ID NOs: 1-3, 5-12, 27-30, and 32-33. SEQ ID NOs: 1-3, 5-12, 27-30, and 32-33 are provided in Table 8 below.

In some embodiments of the present invention, the laundry detergent comprises a peptide, wherein the peptide comprises an amino acid sequence having at least 70%, at least 75%, at least 80%, at least 85%, at least 90%, at least 92%, at least 95%, at least 97%, at least 99%, or 100% identity to any of SEQ ID NOs: 27-33. In some aspects, the peptide has the amino acid sequence of any of SEQ ID NOs: 27-33.

In some aspects, peptides for use in liquid detergents according to the disclosure can further comprise a linking domain. In some aspects, the peptides comprise one or more linking domains. In some aspects, the peptides comprise more than one linking domain. In some aspects, the peptide comprises one or two linking domains. In some aspects, the peptide comprises two linking domains. In some aspects, a linking domain according to the disclosure includes an amino acid sequence of at least four amino acids or at least five amino acids. In some aspects, the linking domain comprises the sequence of GGGS or GGGGS. In some aspects, the linking domains comprise, consist essentially of, or consist of a GGGGS sequence and/or an EAAAK sequence. Exemplary linking domains include, but are not limited to, SEQ ID NO: 13-26, provided in Table 8 below. In some aspects, the one or more linking domains of the peptide have an amino acid sequence having at least 70%, at least 75%, at least 80%, at least 85%, at least 90%, at least 95%, or 100% identity to any one of SEQ ID NO: 13-26. In some aspects, the one or more linking domains have the amino acid sequence of any of SEQ ID NOs: 13-26.

In some aspects, the one or more linking domains flank one or more textile binding domains. In some aspects, the one or more linking domains flank the one or more domains susceptible to oxidation. In some aspects, the one or more linking domains flank the N-terminal end of the textile binding domain and/or the C-terminal end of the textile binding domain. In some aspects, one or more linking domains flank the N-terminal end of the one more domains susceptible to oxidation or the C-terminal end of the one or more domains susceptible to oxidation.

In some aspects, the peptide comprises one or two linking domains. In some aspects, the peptide comprises two linking domains. In some aspects, the textile binding domain is oriented between two linking domains. In some aspects, a linking domain flanks the N-terminal end of the textile binding domain and a second linking domain flanks the C-terminal end of the textile binding domain. In some aspects, a linking domain flanks the C-terminal end of a domain susceptible to oxidation and a second linking domain flanks the N-terminal end of a second domain susceptible to oxidation. In some aspects, the peptide comprises, in N-terminal to C-terminal orientation, a domain susceptible to oxidation, a linking domain, a textile binding domain, a linking domain, and a domain susceptible to oxidation. In some aspects, the two linking domains are the same. In some aspects, the two linking domains are different. In some aspects, the two domains susceptible to oxidation are the same. In some aspects, the two domains susceptible to oxidation are different.

Peptides for use in liquid detergents according to the disclosure have individual amino acids incorporated therein that are susceptible to oxidation. In some aspects, the individual amino acids susceptible to oxidation comprise about 1 to about 75% of the peptide, relative to the total number of amino acids in the peptide. In some aspects, the individual amino acids susceptible to oxidation comprise about 1 to about 50% of the peptide, relative to the total number of amino acids in the peptide. In some aspects, the individual amino acids susceptible to oxidation comprises about 1.5 to about 45% of the peptide, relative to the total number of amino acids in the peptide. In some aspects, the individual amino acids susceptible to oxidation comprises about 2 to about 40% of the peptide, relative to the total number of amino acids in the peptide. In some aspects, the individual amino acids susceptible to oxidation comprises about 2.5 to about 35% of the peptide, relative to the total number of amino acids in the peptide. In some aspects, the amino acids susceptible to oxidation comprises about 3% to about 30% of the peptide, relative to the total number of amino acids in the peptide. In some aspects, the individual amino acids susceptible to oxidation comprises about 3.3% to about 25% of the peptide, relative to the total number of amino acids in the peptide. In some aspects, the individual amino acids susceptible to oxidation comprise about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% of the peptide relative to the total number of amino acids in the peptide.

One of ordinary skill in the art will appreciate that alanine, arginine, asparagine, aspartic acid, cysteine, glutamine, glutamic acid, glycine, histidine, isoleucine, lysine, methionine, phenylalanine, proline, serine, threonine, tryptophan, tyrosine and valine are all susceptible to oxidation in varying degrees. In accordance with various aspect of the disclosure, peptides including one or more of cysteine, methionine, lysine, histidine, and arginine as individual amino acids susceptible to oxidation is preferred. In some aspects, there is at least two individual amino acids susceptible to oxidation within the peptide. In some aspects, there are at least three amino acids susceptible to oxidation within the peptide. In some aspects, there are at least four individual amino acids susceptible to oxidation within the peptide. In some aspects, there are up to 10, alternatively up to 10, alternatively up to 30 individual, alternatively up to 40 individual, and alternatively up to 50 individual amino acids susceptible to oxidation within the peptide. In some aspects, the amount of amino acids susceptible to oxidation within the peptides ranges from about 2 to about 80 amino acids.

In some aspects, the peptides for use in liquid detergents according to the disclosure comprise one or more domains susceptible to oxidation comprising individual amino acids that are susceptible to oxidation. In some aspects, the individual amino acids susceptible to oxidation comprise about 70%, about 75%, about 80%, about 85%, about 90%, about 95%, or 100% of the domain susceptible to oxidation relative to the total number of amino acids in the domain susceptible to oxidation.

In some aspects, the one or more domains susceptible to oxidation comprise:
(1) about 40%, about 45%, or about 50% of the amino acids are M and about 50%, about 55%, or about 60% of the amino acids are selected from Y, D, K, V, A, and E;
(2) about 45%, about 50%, or about 55% of the amino acids are M and about 45%, about 50%, or about 55% of the amino acids are selected from K, Y, and D;
(3) about 65%, about 70%, or about 75% of the amino acids are M and about 25%, about 30%, or about 35% of the amino acids are Y;
(4) about 20%, about 25%, or about 30% of the amino acids are L and about 70%, about 75%, or about 80% of the amino acids are N, G, I, P, Q, F, V, A, and S; or
(5) about 35%, about 40%, or about 45% of the amino acids are M or K and about 55%, about 60%, or about 65% of the amino acids are selected from Y, G, D, V, A, C, S, and E.

In some aspects, the one or more domains susceptible to oxidation comprise:
(1) about 42.5% to about 47.5% of the amino acids are M and about 52.5% to about 57.5% of the amino acids are selected from Y, D, K, V, A, and E;
(2) about 47.5%, to about 52.5% of the amino acids are M and about 47.5%, to about 52.5% of the amino acids are selected from K, Y, and D;
(3) about 70% to about 75% of the amino acids are M and about 25% to about 30% of the amino acids are Y;
(4) about 22.5% to about 27.5% of the amino acids are L and about 72.5% to about 77.5% of the amino acids are N, G, I, P, Q, F, V, A, and S; or
(5) about 37.5% to about 42.5% of the amino acids are M or K and about 57.5% to about 62.5% of the amino acids are selected from Y, G, D, V, A, C, S, and E.

In some aspects, the one or more domains susceptible to oxidation comprise:
(1) about 45% of the amino acids of the one or more domains susceptible to oxidation are M and about 55% of the amino acids are selected from Y, D, K, V, A, and E;
(2) about 50% of the amino acids of the one or more domains susceptible to oxidation are M and about 50% of the amino acids are selected from K, Y, and D;
(3) about 70% of the amino acids of the one or more domains susceptible to oxidation are M and about 30% of the amino acids are Y;
(4) about 25% of the amino acids of the one or more domains susceptible to oxidation are L and about 75% of the amino acids are N, G, I, P, Q, F, V, A, and S; or
(5) about 40% of the amino acids of the one or more domains susceptible to oxidation are selected from M and K and about 60% of the amino acids are selected from Y, G, D, V, A, C, S, and E.

In some aspects, the domain susceptible to oxidation comprises about 40%, about 45%, or about 50% of the amino acids are M and about 50%, about 55%, or about 60% of the amino acids are selected from Y, D, K, V, A, and E. In some aspects, the domain susceptible to oxidation comprises about 42.5% to about 47.5% of the amino acids are M and about 52.5% to about 57.5% of the amino acids are selected from Y, D, K, V, A, and E. In some aspects, the domain susceptible to oxidation comprises about 45% of the amino acids of the one or more domains susceptible to oxidation are M and about 55% of the amino acids are selected from Y, D, K, V, A, and E.

In some aspects, the domain susceptible to oxidation comprises about 45%, about 50%, or about 55% of the amino acids are M and about 45%, about 50%, or about 55% of the amino acids are selected from K, Y, and D. In some aspects, the domain susceptible to oxidation comprises about 47.5%, to about 52.5% of the amino acids are M and about 47.5%, to about 52.5% of the amino acids are selected from K, Y, and D. In some aspects, domain susceptible to oxidation comprises about 50% of the amino acids of the one or more domains susceptible to oxidation are M and about 50% of the amino acids are selected from K, Y, and D.

In some aspects, the domain susceptible to oxidation comprises about 65%, about 70%, or about 75% of the amino acids are M and about 25%, about 30%, or about 35% of the amino acids are Y. In some aspects, the domain susceptible to oxidation comprises about 70% to about 75% of the amino acids are M and about 25% to about 30% of the amino acids are Y. In some aspects, the domain susceptible to oxidation comprises about 70% of the amino acids of the one or more domains susceptible to oxidation are M and about 30% of the amino acids are Y. In some aspects, the domain susceptible to oxidation comprises about 71% of the amino acids of the one or more domains susceptible to oxidation are M and about 28% of the amino acids are Y.

In some aspects, the domain susceptible to oxidation comprises about 20%, about 25%, or about 30% of the amino acids are L and about 70%, about 75%, or about 80% of the amino acids are N, G, I, P, Q, F, V, A, and S. In some aspects, the domain susceptible to oxidation comprises about 22.5% to about 27.5% of the amino acids are L and about 72.5% to about 77.5% of the amino acids are N, G, I, P, Q, F, V, A, and S. In some aspects, the domain susceptible to oxidation comprises about 25% of the amino acids of the one or more domains susceptible to oxidation are L and about 75% of the amino acids are N, G, I, P, Q, F, V, A, and S.

In some aspects, the domain susceptible to oxidation comprises about 35%, about 40%, or about 45% of the amino acids are M or K and about 55%, about 60%, or about 65% of the amino acids are selected from Y, G, D, V, A, C, S, and E. In some aspects, the domain susceptible to oxidation comprises about 37.5% to about 42.5% of the amino acids are M or K and about 57.5% to about 62.5% of the amino acids are selected from Y, G, D, V, A, C, S, and E. In some aspects, the domain susceptible to oxidation comprises about 40% of the amino acids of the one or more domains susceptible to oxidation are selected from M and K and about 60% of the amino acids are selected from Y, G, D, V, A, C, S, and E. In some aspects, the domain susceptible to oxidation comprises about 41% of the amino acids of the one or more domains susceptible to oxidation are selected from M and K and about 59% of the amino acids are selected from Y, G, D, V, A, C, S, and E.

In some aspects, the one or more domains susceptible to oxidation are between about 5 and 25 amino acids in length. In some aspects, the one or more domains susceptible to oxidation are between about 5 and 10 amino acids, about 10 and 15 amino acids, or about 20 and 25 amino acids in length. In some aspects, the one or more domains susceptible to oxidation are about 7, 8, 12, or 22 amino acids in length.

In some aspects, the peptide comprises one or two domains susceptible to oxidation. In some aspects, the peptide comprises two domains susceptible to oxidation. In some aspects, the two domains susceptible to oxidation are the same. In some aspect, the two domains susceptible to oxidation are different.

In some aspects, the peptide comprises two domains susceptible to oxidation that comprise about 40%, about 45%, or about 50% of the amino acids are M and about 50%, about 55%, or about 60% of the amino acids are selected from Y, D, K, V, A, and E. In some aspects, the peptide comprises two domains susceptible to oxidation that comprise about 42.5% to about 47.5% of the amino acids are M and about 52.5% to about 57.5% of the amino acids are selected from Y, D, K, V, A, and E. In some aspects, the peptide comprises two domains susceptible to oxidation that comprise about 45% of the amino acids of the one or more domains susceptible to oxidation are M and about 55% of the amino acids are selected from Y, D, K, V, A, and E.

In some aspects, the peptide comprises two domains susceptible to oxidation that comprise about 55% of the amino acids are M and about 45%, about 50%, or about 55% of the amino acids are selected from K, Y, and D. In some aspects, the peptide comprises two domains susceptible to oxidation that comprise about 47.5%, to about 52.5% of the amino acids are M and about 47.5%, to about 52.5% of the amino acids are selected from K, Y, and D. In some aspects, the peptide comprises two domains susceptible to oxidation that comprise about 50% of the amino acids of the one or more domains susceptible to oxidation are M and about 50% of the amino acids are selected from K, Y, and D.

In some aspects, the peptide comprises two domains susceptible to oxidation that comprise about 65%, about 70%, or about 75% of the amino acids are M and about 25%, about 30%, or about 35% of the amino acids are Y. In some aspects, the peptide comprises two domains susceptible to oxidation that comprise about 70% to about 75% of the amino acids are M and about 25% to about 30% of the amino acids are Y. In some aspects, the peptide comprises two domains susceptible to oxidation that comprise about 70% of the amino acids of the one or more domains susceptible to oxidation are M and about 30% of the amino acids are Y. In some aspects, the peptide comprises two domains susceptible to oxidation that comprise about 71% of the amino acids of the one or more domains susceptible to oxidation are M and about 28% of the amino acids are Y.

In some aspects, the peptide comprises two domains susceptible to oxidation that comprise about 20%, about 25%, or about 30% of the amino acids are L and about 70%, about 75%, or about 80% of the amino acids are N, G, I, P, Q, F, V, A, and S. In some aspects, the peptide comprises two domains susceptible to oxidation that comprise about 22.5% to about 27.5% of the amino acids are L and about 72.5% to about 77.5% of the amino acids are N, G, I, P, Q, F, V, A, and S. In some aspects, the peptide comprises two domains susceptible to oxidation that comprise about 25% of the amino acids of the one or more domains susceptible to oxidation are L and about 75% of the amino acids are N, G, I, P, Q, F, V, A, and S.

In some aspects, the peptide comprises two domains susceptible to oxidation that comprise about 35%, about 40%, or about 45% of the amino acids are M or K and about 55%, about 60%, or about 65% of the amino acids are selected from Y, G, D, V, A, C, S, and E. In some aspects, the peptide comprises two domains susceptible to oxidation that comprise about 37.5% to about 42.5% of the amino acids are M or K and about 57.5% to about 62.5% of the amino acids are selected from Y, G, D, V, A, C, S, and E. In some aspects, the peptide comprises two domains susceptible to oxidation that comprise about 40% of the amino acids of the one or more domains susceptible to oxidation are selected from M and K and about 60% of the amino acids are selected from Y, G, D, V, A, C, S, and E. In some aspects, the peptide comprises two domains susceptible to oxidation that comprise about 41% of the amino acids of the one or more domains susceptible to oxidation are selected from M and K and about 59% of the amino acids are selected from Y, G, D, V, A, C, S, and E.

In some aspects, the one or more domains susceptible to oxidation comprise an amino acid sequence having at least 70%, at least 75%, at least 80%, at least 85%, at least 90%, at least 92%, at least 95%, at least 97%, at least 99%, or 100% identity to any of SEQ ID NOs: 34-38. In some aspects, the one or more domains susceptible to oxidation comprises the amino acid sequence of any of SEQ ID NOs: 34-38.

In some aspects, the peptide comprises two domains susceptible to oxidation comprising an amino acid sequence having at least 70%, at least 75%, at least 80%, at least 85%, at least 90%, at least 92%, at least 95%, at least 97%, at least 99%, or 100% identity to any of SEQ ID NOs: 34-38. In some aspects, the peptide comprises two domains susceptible to oxidation comprising the amino acid sequence of any of SEQ ID NOs: 34-38.

In some aspects, the peptide comprises two domains susceptible to oxidation comprising amino acid sequence having at least 70%, at least 75%, at least 80%, at least 85%, at least 90%, at least 92%, at least 95%, at least 97%, at least 99%, or 100% identity to SEQ ID NO: 34. In some aspects, the peptide comprises two domains susceptible to oxidation comprising the amino acid sequence of SEQ ID NO: 34.

In some aspects, the peptide comprises two domains susceptible to oxidation comprising amino acid sequence having at least 70%, at least 75%, at least 80%, at least 85%, at least 90%, at least 92%, at least 95%, at least 97%, at least 99%, or 100% identity to SEQ ID NO: 35. In some aspects, the peptide comprises two domains susceptible to oxidation comprising the amino acid sequence of SEQ ID NO: 35.

In some aspects, the peptide comprises two domains susceptible to oxidation comprising amino acid sequence having at least 70%, at least 75%, at least 80%, at least 85%, at least 90%, at least 92%, at least 95%, at least 97%, at least 99%, or 100% identity to SEQ ID NO: 36. In some aspects, the peptide comprises two domains susceptible to oxidation comprising the amino acid sequence of SEQ ID NO: 36.

In some aspects, the peptide comprises two domains susceptible to oxidation comprising amino acid sequence having at least 70%, at least 75%, at least 80%, at least 85%, at least 90%, at least 92%, at least 95%, at least 97%, at least 99%, or 100% identity to SEQ ID NO: 37. In some aspects, the peptide comprises two domains susceptible to oxidation comprising the amino acid sequence of SEQ ID NO: 37.

In some aspects, the peptide comprises two domains susceptible to oxidation comprising amino acid sequence having at least 70%, at least 75%, at least 80%, at least 85%, at least 90%, at least 92%, at least 95%, at least 97%, at least 99%, or 100% identity to SEQ ID NO: 38. In some aspects, the peptide comprises two domains susceptible to oxidation comprising the amino acid sequence of SEQ ID NO: 38.

Peptides for use in liquid detergents according to the disclosure include one or more domains having amino acids that exhibit a binding affinity to textiles. In some aspects, the amino acids that bind to textiles are leucine, glycine, alanine, serine, arginine, isoleucine, glutamine, and proline. In some aspects, some amino acids can be both susceptible to oxidation and prone to textile binding. In some aspects, the amino acids exhibiting textile binding affinity comprise about 10% to about 80%, of the peptide, relative to the total number of amino acids in the peptide. In some aspects, the amino acids exhibiting textile binding affinity comprise about 10%, about 15%, about 20%, about 25%, about 30%, about 35%, or about 40% of the peptide, relative to the total number of amino acids in the peptide. In some aspects, the amino acids exhibiting textile binding affinity comprise about 75% or about 80% of the peptide, relative to the total number of amino acids in the peptide. In some aspects, the amino acids exhibiting textile binding affinity comprise about 1 to about 75% of the peptide, relative to the total number of amino acids in the peptide. In some aspects, the amino acids exhibiting textile binding affinity comprise about 1 to about 50% of the peptide, relative to the total number of amino acids in the peptide. In some aspects, the amino acids exhibiting textile binding affinity comprise about 1.5 to about 45% of the peptide, relative to the total number of amino acids in the peptide. In some aspects, the amino acids exhibiting textile binding affinity comprise about 2 to about 40% of the peptide, relative to the total number of amino acids in the peptide. In some aspects, the amino acids exhibiting textile binding affinity comprise about 2.5 to about 35% of the peptide, relative to the total number of amino acids in the peptide. In some aspects, the amino acids exhibiting textile binding affinity comprise about 3% to about 30% of the peptide, relative to the total number of amino acids in the peptide, relative to the total number of amino acids in the peptide. In some aspects, the amino acids exhibiting textile binding affinity comprise about 3.3% to about 25% of the peptide, relative to the total number of amino acids in the peptide. In some aspects, there is 1 amino acid capable of binding to textiles within the peptide.

In some aspects, the peptides for use in liquid detergents according to the disclosure one or more textile binding domains that comprise amino acids that exhibit a binding affinity to textiles. In some aspects, the amino acids exhibiting textile binding affinity comprise about 70%, about 75%, or about 80% of the textile binding domain relative to the total number of amino acids in the textile binding domain.

In some aspects, peptides according to various aspects of the disclosure, that is peptides having one of more domains with amino acids that exhibit a binding affinity to textiles, exhibit a 10-fold, or in some aspects, 20-fold, 50-fold, or 100-fold higher incidence of adhesion to a given textile than alternatives peptides of comparable length without domains exhibit textile binding affinity.

In some aspects, one or more amino acids in the peptide can be both susceptible to oxidation and prone to binding to a textile. In some aspects, an amino acid that is both susceptible to oxidation and prone to binding to a textile is arginine. In some aspects, amino acids that do not have a primary amine group after forming a peptide bond can make up less than 90% of a binding domain of the one or more binding domains. In some aspects amino acids that do not have a primary amine group after forming a peptide bond can make up less than 80% of a binding domain of the one or more binding domains. In some aspects, amino acids that do not have a primary amine group after forming a peptide bond can make up less than 70% of a binding domain of the one or more binding domains. In some aspects, amino acids that do not have a primary amine group after forming the peptide bond can make up less than 60%, 50%, 40%, 30%, 20%, or 10% of a binding domain of the one or more binding domains.

In some aspects, the one or more textile binding domains comprise an amino acid sequence having at least 70%, at least 75%, at least 80%, at least 85%, at least 90%, at least 95%, at least 97%, at least 99%, or 100% identity to SEQ ID NO: 4. In some aspects, the one or more textile binding domains comprise the sequence of SEQ ID NO: 4.

In some aspects, the peptide comprises one or two textile binding domains. In some aspects, the peptide comprises one textile binding domain.

In some aspects, the textile binding domain is oriented between two domains susceptible to oxidation. In some aspects, the peptide comprises a domain susceptible to oxidation flanking the N-terminal end of the textile binding domain or the C-terminal end of the textile binding domain. In some aspects, the peptide comprises a domain susceptible to oxidation flanking the N-terminal end of the textile binding domain and a second domain susceptible to oxidation flanking the C-terminal end of the textile binding domain. In some aspects, the two domains susceptible to oxidation are the same. In some aspects, the two domains susceptible to oxidation are different.

In some aspects, the textile binding domains is oriented between two linking domains. In some aspects, the peptide comprises a linking domain flanking the N-terminal end of the textile binding domain and/or the C-terminal end of the textile binding domain. In some aspects, the peptide comprises a linking domain flanking the N-terminal end of the textile binding domain and a second linking domain flanking the C-terminal end of the textile binding domain. In some aspects, the two linking domains are the same. In some aspects, the two linking domains are different.

In some aspects, the peptide comprises or more domains susceptible to oxidation comprising an amino acid sequence having at least 70%, at least 75%, at least 80%, at least 85%, at least 90%, at least 95%, at least 97%, at least 99%, or 100% identity to SEQ ID NOs: 34-38 and one or more textile binding domains comprising an amino acid sequence having at least 70%, at least 75%, at least 80%, at least 85%, at least 90%, at least 95%, at least 97%, at least 99%, or 100% identity to SEQ ID NO: 4. In some aspects, the peptide comprises one or more domains susceptible to oxidation comprising the amino acid sequence of any of SEQ ID NOs: 34-38 and one or more textile binding domains comprising an amino acid sequence of SEQ ID NO: 4.

In some aspects, the peptide comprises or more domains susceptible to oxidation comprising an amino acid sequence having at least 70%, at least 75%, at least 80%, at least 85%, at least 90%, at least 95%, at least 97%, at least 99%, or 100% identity to SEQ ID NOs: 34-38, one or more textile binding domains comprising an amino acid sequence having at least 70%, at least 75%, at least 80%, at least 85%, at least 90%, at least 95%, at least 97%, at least 99%, or 100% identity to SEQ ID NO: 4, and one or more linking domains comprising an amino acid sequence having at least 70%, at least 75%, at least 80%, at least 85%, at least 90%, at least 95%, at least 97%, at least 99%, or 100% identity to SEQ ID NOs: 13-26. In some aspects, the peptide comprises one or more domains susceptible to oxidation comprising the amino acid sequence of any of SEQ ID NOs: 34-38, one or more textile binding domains comprising an amino acid sequence of SEQ ID NO: 4, and one or more linking domains comprising the amino acid sequence of SEQ ID NOs: 13-26.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NOs: 34-38 and a textile binding domain comprising an amino acid sequence having at least 70%, at least 75%, at least 80%, at least 85%, at least 90%, at least 95%, at least 97%, at least 99%, or 100% identity to SEQ ID NO: 4. In some aspects, the peptide comprises two domains susceptible to oxidation comprising the amino acid sequence of any of SEQ ID NOs: 34-38 and a textile binding domain comprising an amino acid sequence of SEQ ID NO: 4.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NOs: 34-38, a textile binding domain comprising an amino acid sequence having at least 70%, at least 75%, at least 80%, at least 85%, at least 90%, at least 95%, at least 97%, at least 99%, or 100% identity to SEQ ID NO: 4, and two linking domains comprising an amino acid sequence having at least 70%, at least 75%, at least 80%, at least 85%, at least 90%, at least 95%, at least 97%, at least 99%, or 100% identity to SEQ ID NOs: 13-26. In some aspects, the peptide comprises two domains susceptible to oxidation comprising the amino acid sequence of any of SEQ ID NOs: 34-38, a textile binding domain comprising an amino acid sequence of SEQ ID NO: 4, and two linking domains comprising the amino acid sequence of SEQ ID NOs: 13-26.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 34 and a textile binding domain comprising an amino acid sequence having at least 70%, at least 75%, at least 80%, at least 85%, at least 90%, at least 95%, at least 97%, at least 99%, or 100% identity to SEQ ID NO: 4. In some aspects, the peptide comprises two domains susceptible to oxidation comprising the amino acid sequence of SEQ ID NO: 34 and a textile binding domain comprising the amino acid sequence of SEQ ID NO: 4.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 34, a textile binding domain comprising an amino acid sequence having at least 70%, at least 75%, at least 80%, at least 85%, at least 90%, at least 95%, at least 97%, at least 99%, or 100% identity to SEQ ID NO: 4, a linking domain comprising an amino acid sequence having at least 70%, at least 75%, at least 80%, at least 85%, at least 90%, at least 95%, at least 97%, at least 99%, or 100% identity to SEQ ID NO: 25, and a linking domain comprising an amino acid sequence having at least 70%, at least 75%, at least 80%, at least 85%, at least 90%, at least 95%, at least 97%, at least 99%, or 100% identity to SEQ ID NO: 26. In some aspects, the peptide comprises two domains susceptible to oxidation comprising the amino acid sequence of SEQ ID NO: 34, a textile binding domain comprising the amino acid sequence of SEQ ID NO: 4, a linking domain comprising the amino acid sequence of SEQ ID NO: 25, and a linking domain comprising the amino acid sequence of SEQ ID NO: 26.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 35 and a textile binding domain comprising an amino acid sequence having at least 70%, at least 75%, at least 80%, at least 85%, at least 90%, at least 95%, at least 97%, at least 99%, or 100% identity to SEQ ID NO: 4. In some aspects, the peptide comprises two domains susceptible to oxidation comprising the amino acid sequence of SEQ ID NO: 35 and a textile binding domain comprising the amino acid sequence of SEQ ID NO: 4.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 36 and a textile binding domain comprising an amino acid sequence having at least 70%, at least 75%, at least 80%, at least 85%, at least 90%, at least 95%, at least 97%, at least 99%, or 100% identity to SEQ ID NO: 4. In some aspects, the peptide comprises two domains susceptible to oxidation comprising the amino acid sequence of SEQ ID NO: 36 and a textile binding domain comprising the amino acid sequence of SEQ ID NO: 4.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 37, a textile binding domain comprising an amino acid sequence having at least 70%, at least 75%, at least 80%, at least 85%, at least 90%, at least 95%, at least 97%, at least 99%, or 100% identity to SEQ ID NO: 4, and two linking domains comprising an amino acid sequence having at least 70%, at least 75%, at least 80%, at least 85%, at least 90%, at least 95%, at least 97%, at least 99%, or 100% identity to SEQ ID NO: 13. In some aspects, the peptide comprises two domains susceptible to oxidation comprising the amino acid sequence of SEQ ID NO: 37, a textile binding domain comprising the amino acid sequence of SEQ ID NO: 4, and two linking domains comprising the amino acid sequence of SEQ ID NO: 13.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 38, a textile binding domain comprising an amino acid sequence having at least 70%, at least 75%, at least 80%, at least 85%, at least 90%, at least 95%, at least 97%, at least 99%, or 100% identity to SEQ ID NO: 4, a linking domain comprising an amino acid sequence having at least 70%, at least 75%, at least 80%, at least 85%, at least 90%, at least 95%, at least 97%, at least 99%, or 100% identity to SEQ ID NO: 25, and a linking domain comprising an amino acid sequence having at least 70%, at least 75%, at least 80%, at least 85%, at least 90%, at least 95%, at least 97%, at least 99%, or 100% identity to SEQ ID NO: 26. In some aspects, the peptide comprises two domains susceptible to oxidation comprising the amino acid sequence of SEQ ID NO: 38, a textile binding domain comprising the amino acid sequence of SEQ ID NO: 4, a linking domain comprising the amino acid sequence of SEQ ID NO: 25, and a linking domain comprising the amino acid sequence of SEQ ID NO: 26.

In some aspects, the peptide comprises two domains susceptible to oxidation comprising an amino acid sequence having at least 70%, at least 75%, at least 80%, at least 85%, at least 90%, at least 95%, at least 97%, at least 99%, or 100% identity to SEQ ID NO: 38 and a textile binding domain comprising an amino acid sequence having at least 70%, at least 75%, at least 80%, at least 85%, at least 90%, at least 95%, at least 97%, at least 99%, or 100% identity to SEQ ID NO: 4. In some aspects, the peptide comprises two domains susceptible to oxidation comprising the amino acid sequence of SEQ ID NO: 38 and a textile binding domain comprising the amino acid sequence of SEQ ID NO: 4.

In some aspects, each amino acid susceptible to oxidation is no more than one amino acid away from an amino acid exhibiting textile binding affinity in the peptide. In some aspects, each amino acid susceptible to oxidation is no more than two amino acids away from an amino acid exhibiting textile binding affinity in the peptide. In some aspects, each amino acid susceptible to oxidation is no more than 3, 4, 5, 6, 7, 8, 9, 10, 11, 12, 13, 14, 15, 16, 17, 18, 19, 20, or 21 amino acids away from an amino acid exhibiting textile binding affinity in the peptide. In some aspects, a linking domain as described elsewhere herein is located between amino acids susceptible to oxidation and amino acids exhibiting textile binding affinity.

Peptides according to various aspects of the disclosure may be chemically synthesized and/or recombinantly produced by protein design. Short peptides can be easily reproduced synthetically, e.g. via solid-phase synthesis such as Merrifield's solid-phase synthesis. Longer peptides and polypeptides, on the other hand, are often produced recombinantly in the host organism. In some aspects, it may be preferable to produce the peptides according to the disclosure by means of recombinant processes. This is to be understood as any genetic engineering or microbiological process that is based on the introduction of the genes for the peptides of interest into a host organism suitable for production and transcribed and translated by it. Using common methodologies, such as chemical synthesis or polymerase chain reaction (PCR) in conjunction with standard molecular biological and/or protein chemical methods, it is possible to produce the corresponding nucleic acids up to complete genes on the basis of known DNA and/or amino acid sequences.

Peptides according to the invention may exhibit amino acid changes, in particular amino acid substitutions, insertions or deletions. Such peptides have been further developed, e.g. by targeted genetic modification, i.e. by mutagenesis methods, and optimized for specific applications or with regard to special properties (e.g. with regard to their stability, binding, etc.).

For example, targeted mutations such as substitutions, insertions or deletions can be introduced into the known molecules in order to change certain properties. For this purpose, the surface charges and/or the isoelectric point of the molecules and thus their interactions with a surface can be changed. For example, the net charge of the peptides can be changed in order to influence the substrate binding. Alternatively or additionally, one or more corresponding mutations can increase the stability or adsorption of the peptide. Advantageous properties of individual mutations, e.g. individual substitutions, can complement each other.

Thus, the invention also includes peptides characterized by the fact that they are available from a peptide as a starting molecule as described above, e.g. from a molecule with one of the amino acid sequences according to SEQ ID NO:1-3, 5-12, 27-30, and 32-33, on which e.g. one or more amino acid substitutions, including one or more conservative amino acid substitutions, have been performed, wherein the resulting peptide is at least 80% and increasingly preferably at least 81%, 82%, 83%, 84%, 85%, 86%, 87%, 88%, 89%, 90%, 91%, 92%, 93%, 94%, 95%, 96%, 97%, 98%, 99% and less than 100% sequence identity with any of the amino acid sequences according to SEQ ID NO: 1-3, 5-12, 27-30, and 32-33. Furthermore, suitable peptides include those sequences disclosed in U.S. patent application Ser. Nos. 19/094,931 and 19/094,883. The disclosures in U.S. patent application Ser. Nos. 19/094,931 and 19/094,883 are incorporated by reference to this application in their entireties.

In some aspects, the peptide according to the invention may also be modified. Modifications can be, for example, coupling the peptide with certain other molecules or chemical groups, e.g. organic (macro) molecules, e.g. via a covalent bond or a linker/spacer via a suitable amino acid of the chain and/or N- and/or C-terminal.

All of the above features and aspects can be realized individually or in any combination.

Furthermore, peptides used in detergent compositions according to the disclosure can also be at least a subunit (module) of a larger peptide or polypeptide, wherein the polypeptide may comprise a multimer of the sequences described herein, e.g. 1 to 30 repeats, further preferably 2 to 15 repetitions, especially preferably 2 to 10 repetitions, e.g. 2, 3, 4, 5 or 6 repetitions of the peptide. The polypeptide may include or consist of such multimers. In this context, the term “polypeptide” refers in particular to those peptides that comprise 100 or more amino acids. The term “larger peptides” prefers peptides with at least 40 amino acids, unless otherwise described.

In various embodiments, the peptide is a peptide, larger peptide, or polypeptide (multimer) comprising two or more of the peptides or larger peptides as described herein. In different embodiments, the two or more peptides or larger peptides may be connected to each other by at least one spacer, preferably the at least one spacer comprises or consists of 1 to 10 amino acid residues, in particular 2, 3 or 4 amino acid residues, preferably selected from the group consisting of G, P, I, A and S or combinations thereof, in particular GPI or GAS. In such embodiments, the individual peptides are optionally linearly connected to each other via peptide bonds, possibly also via a spacer.

Peptides used in detergent compositions according to the disclosure can be chemically synthesized and/or recombinantly produced by protein design in various embodiments. Nowadays, short peptides can be easily reproduced synthetically, e.g. via solid-phase synthesis such as Merrifield's solid-phase synthesis. Longer peptides and polypeptides, on the other hand, are often produced recombinantly in the host organism, e.g. in bacteria or yeasts.

In some aspects, it may be preferable to produce the peptides and/or peptide conjugates according to the invention by means of recombinant processes. This is to be understood as any genetic engineering or microbiological process that is based on the introduction of the genes for the peptides of interest into a host organism suitable for production and transcribed and translated by it (summarized in the context of this invention as biotechnological processes).

In particular, peptides and/or peptide conjugates according to the disclosure can be produced as polypeptides (multimers) and subsequently cleaved into the functional peptides and/or peptide conjugates. Particularly preferred multimers have 1 to 30 peptide units (each according to the invention), each separated from each other by spacers of 1 to 10 amino acids in length (e.g. 1, 2, 3 or 4 amino acids). Alternatively, the spacers can also be or include interfaces for specific proteases/peptidases, in particular endopeptidases, or form such an interface together with parts of the peptide.

Using methods commonly known in the art, such as chemical synthesis or polymerase chain reaction (PCR) in conjunction with standard molecular biological and/or protein chemical methods, it is possible to produce the corresponding nucleic acids up to complete genes on the basis of known DNA and/or amino acid sequences.

In some aspects, production of the peptides and/or peptide conjugates described herein by biotechnological processes, as described above, may be preferred.

### Detergent Formulations

Certain aspects of the disclosure are directed to liquid detergent formulations comprising the peptides disclosed herein. In some aspects, the liquid detergent comprises a peptide comprising one or more textile binding domains and one or more domains susceptible to oxidation. In some aspects, the liquid detergent comprises a peptide comprising one textile binding domain and two domains susceptible to oxidation. In some aspects, the liquid detergent comprises a peptide comprising one or more linking domains. In some aspects, the liquid detergent comprises a peptide comprising one textile binding domain, two domains susceptible to oxidation, and two linking domains.

In some aspects, a peptide according to the various aspects of disclosure can be present in an amount ranging from about 0.01 to about 5 wt % of a liquid detergent composition according various aspects of the disclosure. For example, a peptide according to the various aspects of disclosure can be present in an amount ranging from about 0.01 wt % to about 4.9 wt %, about 0.01 wt % to about 4.5 wt %, about 0.01 wt % to about 4 wt %, about 0.01 wt % to about 3.5 wt %, about 0.01 wt % to about 3 wt %, about 0.01 wt % to about 2.5 wt %, about 0.01 wt % to about 2 wt %, about 0.01 wt % to about 1.5 wt %, about 0.01 wt % to about 1 wt %, about 0.01 wt % to about 0.5 wt %, about 0.01 wt % to about 0.4 wt %, about 0.01 wt % to about 0.3 wt %, about 0.01 wt % to about 0.2 wt %, about 0.01 wt % to about 0.1 wt %, about 0.01 wt % to about 0.05 wt %, about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 2 wt %, about 2.5 wt %, about 3 wt %, about 3.5 wt %, about 4 wt %, about 4.5 wt %, or about 5 wt %, of the liquid detergent composition. In some aspects, a peptide according to the disclosure can be in an amount ranging from about 0.025 to about 4.5 wt %, alternatively from about 0.05 to about 4 wt %, alternatively from about 0.075 to about 3.5 wt %, alternatively from about 0.1 to about 3 wt %, alternatively from about 0.12 to about 2.5 wt %, alternatively from about 0.13 to about 2 wt %, alternatively from about 0.14 to about 1.5 wt %, and alternatively from about 0.15 to about 1 wt % of a liquid detergent composition according to the disclosure.

In some aspects, an anionic surfactant can be present in an amount ranging from about 1 to about 40 wt % of a liquid detergent composition according to various aspects of the disclosure. For example, in some aspects, the detergent composition comprises an anionic surfactant in an amount of from about 1 wt % to about 40 wt %, from about 1 wt % to about 35 wt %, from about 1 wt % to about 30 wt %, from about 5 wt % to about 25 wt %, from about 5 wt % to about 20 wt %, from about 5 wt % to about 15 wt %, from about 5 wt % to about 10 wt %, about 5 wt %, about 10 wt %, about 15 wt %, about 20 wt %, about 25 wt %, about 30 wt %, about 35 wt %, or about 40 wt %, of the liquid detergent composition. In some aspects, the anionic surfactant can be present in an amount ranging from about 5 to about 40 wt % of a liquid detergent composition according to various aspects of the disclosure. In some aspects, the anionic surfactant is in an amount from 1 wt % up to 5, 7.5, 10, 12.5, 15, 17.5, 20, 22.5, 25, 27.5, 30, 32.5, or 35 wt % of a liquid detergent composition according to the disclosure.

Various classes of anionic surfactants may be used. In some aspects, the anionic surfactant is a linear alkyl sulfonate (LAS) or a linear alkylbenzene sulfonate (LABS). LAS and LABS are water soluble salts between 8 and 22 carbon atoms in the alkyl group. In some aspects, suitable LAS and/or LABS compounds may include salts of C₈-C₁₈ alkyl sulfonic acids and salts of C₈-C₁₈ alkylbenzyl sulfonic acids. Suitable in some aspects, the anionic surfactant is a linear alkyl ether (or laureth) sulfonate. In some aspects, suitable linear alkyl ether sulfonates include a linear C₈-C₁₈ alkyl chain, 4-9 repeating ethylene oxide units, and an anionic head group made up of the sulfonate group and a counter cation. Suitable counter cations for LAS, LABS and linear alkyl ether sulfonates include, but are not necessarily limited to, Na⁺, K⁺, and NH₄⁺. In some aspects, the anionic surfactant is sodium or potassium lauryl sulfate or a sodium or potassium lauryl ether sulfate. In some aspects, the detergent composition comprises more than one anionic surfactant.

In some aspects, detergent compositions according to various aspects of the disclosure may include a linear alkyl benzene sulfonic acid. In some aspects, the linear alkyl benzene sulfonic acid is in an amount ranging from about 3 to about 15 wt % of the detergent composition. In some aspects, the linear alkyl benzene sulfonic acid is in an amount ranging from about 5 to about 10 wt % of the detergent composition.

In some aspects, detergent compositions according to various aspects of the disclosure may include a sodium lauryl sulfate. In some aspects, the sodium lauryl sulfate is in an amount ranging from about 1 to about 15 wt % of the detergent composition. In some aspects, the sodium lauryl sulfate is in an amount ranging from about 2 to about 10 wt % of the detergent composition.

In some aspects, the detergent composition comprises from about 1 wt % to about 35 wt % of a nonionic surfactant, relative to the total weight of the detergent composition. For example, the total non-ionic surfactant content can be from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, about 50 wt %, about 45 wt %, about 40 wt %, about 35 wt %, about 30 wt %, about 25 wt %, about 20 wt %, about 15 wt %, about 10 wt %, about 5 wt %, or about 1 wt %, of the total weight of the detergent composition. In some aspects, the nonionic surfactant is in an amount ranging from about 7.5 to about 25 wt %, alternatively from about 10 to about 20 wt %, alternatively from about 15 to about 20 wt %, and alternatively from about 16 to about 18 wt % of a liquid detergent composition according to the disclosure.

Various nonionic surfactants may be used. Exemplary nonionic surfactants include, but are not limited to, alkoxylated alcohols, polyoxyalkylene alkyl ethers, polyoxyalkylene alkylphenyl ethers, polyoxyalkylene sorbitan fatty acid esters, polyoxyalkylene sorbitol fatty acid esters, polyalkylene glycol fatty acid esters, alkyl polyalkylene glycol fatty acid esters, polyoxyethylene polyoxypropylene alkyl ethers, polyoxyalkylene castor oils, polyoxyalkylene alkylamines, glycerol fatty acid esters, alkylglucosamides, alkylglucosides, alkylamine oxides, or any combinations thereof.

In some aspects, a combined amount of nonionic surfactant and anionic surfactant in liquid detergent compositions according to various aspects of the disclosure can be in an amount ranging from about 1 wt % to about 60 wt %. For example, a combined amount of nonionic surfactant and anionic surfactant can be from about 1 wt % to about 60 wt %, about 1 wt % to about 55 wt %, about 1 wt % to about 50 wt %, about 1 wt % to about 45 wt %, about 1 wt % to about 40 wt %, about 1 wt % to about 35 wt %, about 1 wt % to about 30 wt %, about 1 wt % to about 25 wt %, about 1 wt % to about 20 wt %, about 1 wt % to about 15 wt %, about 1 wt % to about 5 wt %, about 60 wt %, about 50 wt %, about 40 wt %, about 30 wt %, about 29 wt %, about 28 wt %, about 27 wt %, about 26 wt %, about 25 wt %, about 24 wt %, about 23 wt %, about 22 wt %, about 21 wt %, about 20 wt %, about 19 wt %, about 18 wt %, about 17 wt %, about 16 wt %, about 15 wt %, about 14 wt %, about 13 wt %, about 12 wt %, about 11 wt %, about 10 wt %, about 5 wt %, or about 1 wt %, of the total weight of the detergent composition. In some aspects, a combined amount of nonionic surfactant and anionic surfactant in liquid detergent compositions according to various aspects of the disclosure can be in an amount ranging from about 5 to about 22.5 wt %, alternatively from about 10 to about 20 wt %, alternatively from about 15 to about 20 wt %, and alternatively from about 16 to about 18 wt %. In some aspects, the combined amount of nonionic surfactant and anionic surfactant in liquid detergent compositions according to various aspects of the disclosure can be in an amount ranging from about 1.5 to about 45 wt %, alternatively from about 2 to about 40 wt %, alternatively from about 2.5 to about 35 wt %, and alternatively from about 3 to about 35 wt %.

In some aspects, liquid detergent compositions according to various aspects of the disclosure have a nonionic surfactant to anionic surfactant ration ranging from about 5:1 to about 1:5. In some aspects, the ratio of nonionic surfactants to anionic surfactant can range from about 4:1 to about 1:4, alternatively from about 3:1 to about 1:3, alternatively from about 2:1 to about 1:2, alternatively from about 1.5:1 to about 1:1.5, and alternatively about 1:1. In some aspects, the ratio of nonionic surfactants to anionic surfactant can range from about 10:1 to about 1:10, alternatively from about 9:1 to about 1:9, alternatively from about 8:1 to about 1:8, alternatively from about 7:1 to about 1:7, and alternatively from about 6:1 to about 1:6.

Detergent compositions according to various aspects of the disclosure can have pH values ranging from about 5 to 12, alternatively from about 5.5 to about 12, alternatively, alternatively from about 6 to about 12, alternatively from about 6.5 to about 12, alternatively from about 9 to about 12, alternatively from about 7 to about 10, alternatively from about 10 to about 12, alternatively from about 7 to about 12, and alternatively from about 7 to about 11.5. For example, when such detergent compositions include one or more enzymes, as discussed further below, the pH value may optimally range from about 7 to about 9 to promote stability of said one or more enzymes. Also, for example, detergent compositions do not contain enzymes can have a pH as high as about 10, alternatively as high as about 10.5, alternatively as high as about 11, and alternatively as high as about 11.5.

In some aspects, detergent compositions according to various aspects of the disclosure may include one or more non-aqueous solvents. As used herein, the term “non-aqueous solvent” is directed towards a broad class of compounds. In some aspects, the non-aqueous solvent is one or a combination of a polyol (i.e., a hydrocarbon having more than one hydroxyl group such as glycerol (glycerin), propylene glycol and ethylene glycol), an alcohol (such as ethanol), and a 4C+ compound. As used herein, a “4C+ compound” refers to an organic compound having 4 or more carbons and at least one oxygen group (for example, an alcohol, ether, ester, alkoxy, and so on). As such, certain 4C+ compounds may also be alcohols or polyols with at least four carbons. In some aspects, suitable 4C+ compounds include, but are not limited to, one or more of: polypropylene glycol; polyethylene glycol esters such as polyethylene glycol stearate, propylene glycol laurate, and/or propylene glycol palmitate; ethyl ester ethoxylate; diethylene glycol; dipropylene glycol; tetramethylene glycol; butylene glycol; pentanediol; hexylene glycol; heptylene glycol; octylene glycol; 2-methyl, 1,3-propanediol; triethylene glycol; polypropylene glycol; glycol ethers, such as ethylene glycol monobutyl ether, diethylene glycol monobutyl ether, triethylene glycol monobutyl ether, ethylene glycol monopropyl ether, diethylene glycol monoethyl ether, triethylene glycol monoethyl ether, diethylene glycol monomethyl ether, and triethylene glycol monomethyl ether; tris(2-hydroxyethyl)methyl ammonium methylsulfate; ethylene oxide/propylene oxide copolymers with a number average molecular weight of 3,500 Daltons or less; and ethoxylated fatty acids.

In some aspects, the non-aqueous solvent is or includes a relatively low molecular weight polyethylene glycol (PEG). In some aspects, the polyethylene glycol has a molecular weight ranging from about 200 to about 1000 g/mol (or Daltons), alternatively a molecular weight ranging from about 200 to about 800 g/mol, or alternatively a molecular weight of about 400 g/mol. In some aspects, the polyethylene glycol has a weight average molecular weight of less than about 600 Da, e.g. about 400, such as those having a weight average molecular weight of from about 380 to about 420 Da. In other aspects, PEG 200, PEG 250, PEG 300, PEG 350, PEG 400, PEG 450, PEG 500, PEG 550, and/or PEG 600 (wherein the numerals represent the approximate weight average molecular weight in Daltons or grams/mol) may be used.

In some aspects, the non-aqueous solvent is or includes an ethylene oxide/propylene oxide block copolymer. In some aspects, the non-aqueous solvent is or includes a polyol such as glycerin. In some aspects, the non-aqueous solvent is or includes a mixture of a polyol and a polyethylene glycol. In some aspects, the polyol in the mixture is glycerin. Suitable polyol/polyethylene glycol mixtures may have a polyol to polyethylene glycol weight:weight ratio ranging from about 10:1 to about 1:10, alternatively from about 9:1 to about 1:9, alternatively from about 8:1 to about 1:8, alternatively from about 7:1 to about 1:7, alternatively from about 6:1 to about 1:6, alternatively from about 5:1 to about 1:5, alternatively from about 4:1 to about 1:4, alternatively from about 3:1 to about 1:3, alternatively from about 2:1 to about 1:2, alternatively from about 1.5:1 to about 1:1.5, and alternatively about 1:1.

In some aspects, the non-aqueous solvent is or includes propylene glycol. In some aspects, the propylene glycol is in an amount ranging from about 1 to about 10 wt % of the detergent composition. In some aspects, the propylene glycol is in an amount ranging from about 2.5 to about 7.5 wt % of the detergent composition. In some aspects, the propylene glycol is in an amount of about 5 wt % of the detergent composition. In some aspects, the propylene glycol is in an amount ranging from about 0.1 to about 10 wt % of the detergent composition.

In some aspects, the non-aqueous solvent is or includes ethanol. In some aspects, the ethanol is in an amount ranging from about 1 to about 10 wt % of the detergent composition. In some aspects, the ethanol is in an amount ranging from about 2.5 to about 7.5 wt % of the detergent composition. In some aspects, the ethanol is in an amount of about 5 wt % of the detergent composition. In some aspects, the ethanol is in an amount ranging from about 0.1 to about 5 wt % of the detergent composition.

In some aspects, the non-aqueous solvent is in an amount ranging from about 5 to about 70 wt % of the detergent composition. For example, the non-aqueous solvent can be present in an amount from about 5 wt % to about 70 wt %, about 10 wt % to about 70 wt %, about 15 wt % to about 70 wt %, about 20 wt % to about 70 wt %, about 25 wt % to about 70 wt %, about 30 wt % to about 70 wt %, about 35 wt % to about 70 wt %, about 40 wt % to about 70 wt %, about 45 wt % to about 70 wt %, about 50 wt % to about 70 wt %, about 55 wt % to about 70 wt %, about 60 wt % to about 70 wt %, about 65 wt % to about 50 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, or about 70 wt %, of the total weight of the detergent composition. In some aspects, the non-aqueous solvent is in an amount ranging from about 15 to about 65% of the detergent composition. In some aspects, the detergent composition comprises more than one non-aqueous solvent. In some aspects, the non-aqueous solvent(s) is in an amount ranging from about 5 to about 40 wt % of the detergent composition. In some aspects, the non-aqueous solvent(s) is in an amount ranging from about 20 to about 50 wt % of the detergent composition. In some aspects, the non-aqueous solvent(s) is in an amount ranging from about 10 to about 30 wt % of the detergent composition. In some aspects, the non-aqueous solvent is or comprises glycerin. In some aspects, the non-aqueous solvent is or comprises glycerin, polyethylene glycol, and ethanol. In some aspects, the non-aqueous solvent is or comprises glycerin, propylene glycol and ethanol. In some aspects, the non-aqueous solvent is or comprises propylene glycol and ethanol.

Mildness is a significant consumer need and driver for laundry detergent purchase. There are several methods to measure the skin irritancy potential of a surfactant composition, such as the testing methods as described in U.S. 2018/0016523, the entire disclosure of which is incorporated herein by reference. These tests include Zein, Corneosufametry, and Cytokine testing, which can be used to determine the mildness of a detergent for consumers.

As used herein the phrase “Zein score” refers the measurement obtained from the Zein test as described herein. It can also be referred to as “Zein solubilized %.” In some aspects, detergent compositions described herein have a Zein score of less than about 3% when tested as a 10% dilution. In certain aspects, detergent compositions described herein have a Zein score of from about 0.01% to about 3%, alternatively from about 0.01% to about 2.75%, alternatively from about 0.01% to about 2.5%, alternatively from about 0.01% to about 2.25%, alternatively from about 0.01% to about 2%, alternatively from about 0.01% to about 1.75%, alternatively from about 0.01% to about 1.5%, alternatively from about 0.01% to about 1.25%, and alternatively from about 0.01% to about 1% when tested as a 10% dilution. In other aspects, detergent compositions described herein can have a Zein score of less than about 2.75%, preferably less than about 2.5%, more preferably less than about 2.25%, even more preferably less than about 2%, even more preferably less than 1.75%, even more preferably less than about 1.5%, even more preferably less than about 1.25%, and even more preferably less than about 1% when tested as a 10% dilution. In some aspects, detergent compositions described herein have a Zein score less than 0.5%. In some aspects, detergent compositions described herein have a Zein score ranging from about 0.3 to about 0.4%.

In some aspects, detergent compositions according to various aspects of the disclosures may exhibit a pH ranging from about 9 to about 12, a zein score of less than about 2%, or both. In some aspects, the pH of such detergent compositions can range from about 7 to about 12. In some aspects, the pH of such detergent compositions can range from about 8 to about 12. In some aspects, the pH of such detergent compositions can range from about 10 to about 12.

In some aspects, detergent compositions according to various aspects of the disclosure may further comprise a suitable tetraborate salt such as, for example, sodium tetraborate. In some aspects, the tetraborate salt is in an amount ranging from about 0.01 to about 2 wt % of the detergent composition. For example, the tetraborate salt can be present in an amount from about 0.01 wt % to about 2 wt %, from about 0.01 wt % to about 1.5 wt %, about 0.01 wt % to about 1 wt %, from about 0.01 wt % to about 0.5 wt %, from about 0.01 wt % to about 0.3 wt %, about 0.01 wt % to about 0.2 wt %, about 0.01 wt % to about 0.1 wt %, about 0.01 wt % to about 0.05 wt %, about 0.01 wt %, about 0.05 wt %, about 1.5 wt %, or about 2 wt %, of the total weight of the detergent composition. In some aspects, a suitable tetraborate salt can be present in an amount ranging from about 0.1 to about 1.0 wt % of a detergent composition. In some aspects, a suitable tetraborate salt can be present in an amount ranging from about 0.025 to about 1.75 wt %, alternatively from about 0.05 to about 1.5 wt %, and alternatively from about 0.075 to about 1.25 wt % of a detergent composition. In some aspects, the tetraborate salt is in an amount of about 0.1, 0.2, 0.3, 0.4, 0.5, 0.6, 0.7, 0.8, 0.9, or 1.0 wt % of a detergent composition.

In some aspects, detergent compositions according to various aspects of the disclosure may further comprise a strong base. In some aspects, the strong base is in an amount ranging from about 0.1 wt % to about 10 wt % of base relative to the total weight of the composition. For example, the base can be present in an amount from about 0.1 wt % to about 10 wt %, 0.5 wt % to about 10 wt %, 1 wt % to about 10 wt %, about 1 wt % to about 8 wt %, about 1 wt % to about 5 wt %, about 1 wt % to about 3 wt %, about 1.5 wt % to about 10 wt %, about 1.5 wt % to about 8 wt %, about 1.5 wt % to about 5 wt %, about 1.5 wt % to about 3 wt %, about 2 wt % to about 10 wt %, about 2 wt % to about 8 wt %, about 2 wt % to about 5 wt %, about 2 wt % to about 3 wt %, about 0.1 wt %, about 0.5 wt %, about 1 wt %, about 1.1 wt %, about 1.2 wt %, about 1.3 wt %, about 1.4 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, or about 10 wt %, of the total weight of the composition. In some aspects, the strong base is in an amount ranging from about 0.5 to about 5 wt % of the detergent composition. In some aspects, the amount of the strong base is about 2 wt % to about 3 wt % of the detergent composition. In some aspects, the strong base can be, for example, potassium hydroxide, sodium hydroxide, or calcium hydroxide. Generally, any amount of strong base may be added to a detergent composition to provide said detergent composition with a target end property such as, for example, pH.

In some aspects, detergent compositions according to various aspects of the disclosure may further comprise a weak acid. In some aspects, the weak acid is in an amount ranging from about 0.5 to about 9 wt % of the detergent composition. For example, the weak acid can be present in an amount from about 0.5 wt % to about 9 wt %, about 0.5 wt % to about 8.5 wt %, about 0.5 wt % to about 8 wt %, about 0.5 wt % to about 7.5 wt %, about 0.5 wt % to about 7 wt %, 0.5 wt % to about 6.5 wt %, about 0.5 wt % to about 6 wt %, about 0.5 wt % to about 5.5 wt %, about 0.5 wt % to about 5 wt %, about 0.5 wt % to about 4.5 wt %, about 0.5 wt % to about 4 wt %, 0.5 wt % to about 3.5 wt %, about 0.5 wt % to about 3 wt %, about 0.5 wt % to about 2.5 wt %, about 0.5 wt % to about 2 wt %, about 0.5 wt % to about 1.5 wt %, about 0.5 wt % to about 1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, or about 9 wt % of the total weight of the composition. In some aspects, the weak acid can be, for example, citric acid, acetic acid, lactic acid, stearic acid, palmitic acid, or oleic acid. Generally, any amount of weak acid may be added to a detergent composition to provide said detergent composition with a target end property such as, for example, pH.

In some aspects, detergent compositions according to various aspects of the disclosure may include both a strong acid and a weak acid as described above. Generally any amount of weak acid or strong base may be added to a detergent composition to provide said detergent composition with a target end property such as, for example, pH. In some aspects, the strong base and weak acid are used as a pH adjuster.

In some aspects, detergent compositions according to various aspects of the disclosure may include a fatty acid. In some aspects, the fatty acid is in an amount ranging from about 0.1 to about 9 wt % of the detergent composition. For example, the fatty acid can be present in an amount of from about 0.1 wt % to about 9 wt %, about 0.1 wt % to about 8.5 wt %, about 0.1 wt % to about 8 wt %, about 0.1 wt % to about 7.5 wt %, about 0.1 wt % to about 7 wt %, about 0.1 wt % to about 6.5 wt %, about 0.1 wt % to about 6 wt %, about 0.1 wt % to about 5.5 wt %, about 0.1 wt % to about 5 wt %, about 0.1 wt % to about 4.5 wt %, about 0.1 wt % to about 4 wt %, about 0.1 wt % to about 3.5 wt %, about 0.1 wt % to about 3 wt %, about 0.1 wt % to about 2.5 wt %, about 0.1 wt % to about 2 wt %, about 0.1 wt % to about 1.5 wt %, about 0.1 wt % to about 1 wt %, about 0.1 wt % to about 0.5 wt %, about 0.1 wt %, about 0.5 wt %, about 1 wt %, about 2 wt %, about 3 wt %, about 4 wt %, about 5 wt %, about 6 wt %, about 7 wt %, about 8 wt %, or about 9 wt %, of the total weight of the detergent composition. In some aspects, the fatty acid is a coconut fatty acid. In some aspects, the fatty acid may be one or more of lauric, myristic, palmitic, stearic, ricinoleic, oleic, linoleic, or linolenic acids.

In some aspects, detergent compositions according to various aspects of the disclosure may include an aqueous enzyme solution comprising a cocktail of one or more enzymes. In some aspects, the one or more enzymes may include, for example, one or more of an amylase, a protease, and a mannanase. In some embodiments, protease is excluded from compositions to further stabilize the peptide and thereby improving the efficacy of the peptide. In some aspects, the aqueous enzyme solution is an amount ranging from about 0.1 to about 5 wt % of the detergent composition. For example, the aqueous enzyme solution can be present in an amount of from about 0.1 wt % to about 4.5 wt %, about 0.1 wt % to about 4 wt %, about 0.1 wt % to about 3.5 wt %, about 0.1 wt % to about 3 wt %, about 0.1 wt % to about 2.5 wt %, about 0.1 wt % to about 2 wt %, about 0.1 wt % to about 1.5 wt %, about 0.1 wt % to about 1 wt %, about 0.1 wt % to about 0.8 wt %, about 0.1 wt % to about 0.5 wt %, about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, or about 5 wt %, relative to the total weight of the composition. In some aspects, the aqueous enzyme solution is in an amount ranging from about 1 to about 3 wt % of the detergent composition. In some aspects, the aqueous enzyme solution is in an amount ranging from about 0.5 to about 5 wt % of the detergent composition. In some aspects, the aqueous enzyme solution is in an amount ranging from 0.5 to 2.5 wt % of the detergent composition.

In some aspects, detergent compositions according to various aspects of the disclosure may include a chelating agent. In some aspects, the chelating agent is in an amount ranging from about 0.1 to about 5 wt % of the detergent composition. For example, the chelating agent can be present in an amount of from about 0.1 wt % to about 5 wt %, about 0.1 wt % to about 4.5 wt %, about 0.1 wt % to about 4 wt %, about 0.1 wt % to about 3.5 wt %, about 0.1 wt % to about 3 wt %, about 0.1 wt % to about 2.5 wt %, about 0.1 wt % to about 2 wt %, about 0.1 wt % to about 1.5 wt %, about 0.1 wt % to about 1 wt %, about 0.1 wt % to about 0.8 wt %, about 0.1 wt % to about 0.5 wt %, about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, or about 5 wt %, relative to the total weight of the composition. In some aspects, the chelating agent is N,N-bis(carboxymethyl)-L-glutamic acid tetrasodium salt. Chelating agents are sometimes used as water softeners in detergent compositions. In some aspects, the chelating agent is iminodisuccinate (IDS), ethylenediaminetetraacetic acid (EDTA), nitrilotriacetic acid, diethylenetriaminepenta (methylenephosphonic acid), nitrilotris(methylenephosphonic acid), I-hydroxyethane-1,1-diphosphonic acid, ethylenediamine-N,N′-disuccinic acid (EDDS), hydroxyethylenediaminetriacetic acid (HEDTA), N,N-bis(carboxymethyl)-L-glutamic acid tetrasodium salt, alanine, N,N-bis(carboxymethyl)-alanine, trisodium salt or other chelating compounds.

In some aspects, detergent compositions according to various aspects of the disclosure may include a preservative. In some aspects, the preservative is in an amount ranging from about 0.01 to about 2 wt % of the detergent composition. In some aspects, the detergent composition comprises a preservative in an amount of about 0.01 wt % to about 2 wt %, about 0.05 wt % to about 2 wt %, about 0.1 wt % to about 2 wt %, about 0.5 wt % to about 2 wt %, about 1 wt % to about 2 wt %, about 1.5 wt % to about 2 wt %, about 0.01 wt %, about 0.02 wt %, about 0.04 wt %, about 0.06 wt %, about 0.08 wt %, about 0.1 wt %, about 0.5 wt %, about 0.8 wt %, about 1 wt %, about 1.5 wt %, or about 2 wt % of the detergent composition. In some aspects, the preservative is one or more of a microbiocide, an algicide, and a fungicide. In some aspects, the preservative is a mixture of 5-chloro-2-methyl-2H-isothiazol-3-one and 2-methylisothiazol-3 (2H)-one and 1,2-benzisothiazol-3 (2H)-one. In some aspects, preservative is methylisothiazolinone, chloromethylisothiazolinone, benzisothiazolinone, sorbic acid, sodium benzoate, formaldehyde, borate, and glutaraldehyde. In some aspects, detergent compositions according to various aspects of the disclosure may be free of, or substantially free of, preservatives.

In some aspects, detergent compositions according to various aspects of the disclosure may include an anti-redeposition polymer. In some aspects, anti-redeposition agents include polymers with a soil detachment capacity, which are also known as “soil repellents” due to their ability to provide a soil-repelling finish on the treated surface, such as a fiber. In some aspects, the anti-redeposition polymer is in an amount ranging from about 0.1 to about 3 wt % of the detergent composition. In some aspects, the detergent composition comprises from about 0.1 wt % to about 2.8 wt %, about 0.1 wt % to about 2.5 wt %, about 0.1 wt % to about 2 wt %, about 0.1 wt % to about 1.8 wt %, about 0.1 wt % to about 1.5 wt %, about 0.1 wt % to about 1 wt %, about 0.1 wt % to about 0.8 wt %, about 0.1 wt % to about 0.5 wt %, about 0.1 wt %, about 0.5 wt %, about 0.8 wt %, about 1 wt %, about 1.2 wt %, about 1.5 wt %, about 1.8 wt %, about 2 wt %, about 2.2 wt %, about 2.5 wt %, about 2.8 wt %, or about 3 wt %, of the detergent composition. In some aspects, the anti-redeposition polymer is an acrylic/styrene copolymer. In some aspects, the polymer can be a polyester. In some aspects, the polyesters include co-polyesters prepared from dicarboxylic acids, such as adipic acid, phthalic acid or terephthalic acid. In some aspects, an anti-redeposition agents includes polyesters with a soil detachment capacity that include those compounds which, in formal terms, are obtainable by esterifying two monomer moieties, the first monomer being a dicarboxylic acid HOOC-Ph-COOH and the second monomer a diol HO—(CHR—)ₐOH, which may also be present as a polymeric diol H—(O—(CHR—)ₐ)_{b}OH. “Ph” here means an ortho-, meta- or para-phenylene residue that may bear 1 to 4 substituents selected from alkyl residues with 1 to 22 C atoms, sulfonic acid groups, carboxyl groups and mixtures thereof, “R” means hydrogen or an alkyl residue with 1 to 22 C atoms and mixtures thereof, “a” means a number from 2 to 6 and “b” means a number from 1 to 300. In some aspects, the polyesters obtainable therefrom may contain not only monomer diol units —O—(CHR—)ₐO— but also polymer dial units-(O—(CHR—)ₐ)_{b}O—. In some aspects, the molar ratio of monomer diol units to polymer diol units may amount to from about 100:1 to about 1:100, or alternatively from about 10:1 to about 1:10. In some aspects, the polymer diol units, the degree of polymerization “b” may be in the range of from about 4 to about 200, or alternatively from about 12 to about 140. In some aspects, the number average molecular weight of the polyesters with a soil detachment capacity may be in the range of from about 250 to about 100,000, or alternatively from about 500 to about 50,000. In some aspects, the acid on which the residue Ph is based may be selected from terephthalic acid, isophthalic acid, phthalic acid, trimellitic acid, mellitic acid, the isomers of sulfophthalic acid, sulfoisophthalic acid and sulfoterephthalic acid and mixtures thereof. In some aspects, the acid groups thereof are not part of the ester bond in the polymer, they may be present in salt form, such as an alkali metal or ammonium salt.

In some aspects, instead of the monomer HOOC-Ph-COOH, polyesters with a soil detachment capacity (the anti-redeposition agent) may include small proportions, for example up to about 10 mole percent relative to the proportion of Ph with the above-stated meaning, of other acids that include at least two carboxyl groups. These include, for example, alkylene and alkenylene dicarboxylic acids such as malonic acid, succinic acid, fumaric acid, maleic acid, glutaric acid, adipic acid, pimelic acid, suberic acid, azelaic acid and sebacic acid. Exemplary diols HO—(CHR—)ₐOH include those in which R is hydrogen and “a” is a number of from about 2 to about 6, and in some aspects those in which “a” has the value of 2 and R is selected from hydrogen and alkyl residues with 1 to 10 C atoms, or where R is selected from hydrogen and alkyl residues with 1 to 3 C atoms in another embodiment. Examples of diol components include ethylene glycol, 1,2-propylene glycol, 1,3-propylene glycol, 1,4-butanediol, 1,5-pentanediol, 1,6-hexanediol, 1,8-octanediol, 1,2-decanediol, 1,2-dodecanediol and neopentyl glycol. The polymeric diols may include polyethylene glycol with an average molar mass in the range from about 1000 to about 6000. In some aspects, these polyesters may also be end group-terminated, with end groups that may be alkyl groups with 1 to 22 C atoms or esters of monocarboxylic acids. The end groups attached via ester bonds may be based on alkyl, alkenyl and aryl monocarboxylic acids with 5 to 32 C atoms, or alternatively with 5 to 18 C atoms. These may include valeric acid, caproic acid, enanthic acid, caprylic acid, pelargonic acid, capric acid, undecanoic acid, undecenoic acid, lauric acid, lauroleic acid, tridecanoic acid, myristic acid, myristoleic acid, pentadecanoic acid, palmitic acid, stearic acid, petroselinic acid, petroselaidic acid, oleic acid, linoleic acid, linolaidic acid, linolenic acid, eleostearic acid, arachidic acid, gadoleic acid, arachidonic acid, behenic acid, erucic acid, brassidic acid, clupanodonic acid, lignoceric acid, cerotic acid, melissic acid, benzoic acid, which may bear 1 to 5 substituents having a total of up to 25 C atoms, or alternatively 1 to 12 C atoms, for example tert-butylbenzoic acid. The end groups may also be based on hydroxymonocarboxylic acids with 5 to 22 C atoms, which for example include hydroxyvaleric acid, hydroxycaproic acid, ricinoleic acid, the hydrogenation product thereof, hydroxystearic acid, and ortho-, meta- and para-hydroxybenzoic acid. The hydroxymonocarboxylic acids may in turn be joined to one another via their hydroxyl group and their carboxyl group and thus be repeatedly present in an end group. The number of hydroxymonocarboxylic acid units per end group, i.e. their degree of oligomerization, may be in the range of from 1 to 50, or alternatively in the range of from 1 to 10. In some aspects, polymers of ethylene terephthalate and polyethylene oxide terephthalate, in which the polyethylene glycol units have molar weights of from about 750 to about 5000 and the molar ratio of ethylene terephthalate to polyethylene oxide terephthalate of from about 50:50 to about 90:10, can be used alone or in combination with cellulose derivatives.

In some aspects, detergent compositions according to various aspects of the disclosure may include an optical brightener. Optical brighteners adsorb ultraviolet and/or violet light and re-transmit it as visible light, typically a visible blue light. Optical brighteners include, but are not limited to, derivatives of diaminostilbene disulfonic acid or alkali metal salts thereof. Suitable compounds are, for example, salts of 4,4′-bis(2-anilino-4-morpholino-1,3,5-triazinyl-6-amino) stilbene 2,2′-disulfonic acid or compounds of similar structure which, instead of the morpholino group, bear a diethanolamino group, a methylamino group, an anilino group or a 2-methoxyethylamino group. Optical brighteners of the substituted diphenylstyryl type may furthermore be present, such as the alkali metal salts of 4,4′-bis(2-sulfostyryl)diphenyl, 4,4′-bis(4-chloro-3-sulfostyryl)diphenyl, or 4-(4-chlorostyryl)-4′-(2-sulfostyryl)diphenyl. Mixtures of the above-stated optical brighteners may also be used. In some aspects, the detergent composition comprises from about 0.01 wt % to about 5 wt % of an optical brightener. In some aspects, the detergent composition comprises an optical brightener in and amount of from about 0.01 wt % to about 5 wt %, about 0.01 wt % to about 4.5 wt %, about 0.01 wt % to about 4 wt %, about 0.01 wt % to about 3.5 wt %, about 0.01 wt % to about 3 wt %, about 0.01 wt % to about 2.5 wt %, about 0.01 wt % to about 2 wt %, about 0.01 wt % to about 1.5 wt %, about 0.01 wt % to about 1 wt %, about 0.01 wt % to about 0.8 wt %, about 0.01 wt % to about 0.5 wt %, about 0.01 wt % to about 0.1 wt %, about 0.01 wt %, about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, or about 5 wt %, relative to the total weight of the composition.

In some aspects, detergent compositions according to various aspects of the disclosure may include one or more foam inhibitors (i.e., defoamers). In some aspects, foam inhibitors include, but are not limited to, fatty acids such as coconut fatty acids. In some aspects, suitable foam inhibitors include, for example, soaps of natural or synthetic origin (which may exhibit elevated proportions of C₁₈-C₂₄ fatty acids), organopolysiloxanes and mixtures thereof with microfine (and optionally silanized) silica, paraffins, waxes, microcrystalline waxes and mixtures thereof with silanized silica or bis-fatty acid alkylenediamides (for example, bistearylethylenediamide), silicones, and combinations thereof. In some aspects, the detergent composition comprises from about 0.01 wt % to about 5 wt % of a foam inhibitor. For example, the detergent composition can comprise a foam inhibitor in an amount of from about 0.01 wt % to about 5 wt %, about 0.01 wt % to about 4.5 wt %, about 0.01 wt % to about 4 wt %, about 0.01 wt % to about 3.5 wt %, about 0.01 wt % to about 3 wt %, about 0.01 wt % to about 2.5 wt %, about 0.01 wt % to about 2 wt %, about 0.01 wt % to about 1.5 wt %, about 0.01 wt % to about 1 wt %, about 0.01 wt % to about 0.8 wt %, about 0.01 wt % to about 0.5 wt %, about 0.01 wt % to about 0.1 wt %, about 0.01 wt %, about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, or about 5 wt %, relative to the total weight of the detergent composition.

In some aspects, detergent compositions according to various aspects of the disclosure may include bittering agents to hinder accidental ingestion of the composition. Bittering agents are compositions that taste bad, so children or others are discouraged from accidental ingestion. In some aspects, the bittering agent is denatonium benzoate, aloin, or others. Bittering agents may be present in the composition in an amount ranging from about 0.01 to about 5 wt %. For example, the detergent composition can comprise a bittering agent in an amount of from about 0.01 wt % to about 5 wt %, about 0.01 wt % to about 4.5 wt %, about 0.01 wt % to about 4 wt %, about 0.01 wt % to about 3.5 wt %, about 0.01 wt % to about 3 wt %, about 0.01 wt % to about 2.5 wt %, about 0.01 wt % to about 2 wt %, about 0.01 wt % to about 1.5 wt %, about 0.01 wt % to about 1 wt %, about 0.01 wt % to about 0.8 wt %, about 0.01 wt % to about 0.5 wt %, about 0.01 wt % to about 0.1 wt %, about 0.01 wt %, about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, or about 5 wt %, relative to the total weight of the detergent composition.

In some aspects, detergent compositions according to various aspects of the disclosure may include sodium sulfite. Sodium sulfite is an oxygen scavenger, where sodium sulfite reacts with oxygen to form sodium sulfate. Free oxygen, such as oxygen dissolved in the wash composition, can react to produce metal oxides (rust) that reduce the life of the washing equipment. The metal oxides can also stain garments, dishes, or other items being washed. Dissolved oxygen can also react to produce other components, and some of those components may be colored bodies. Therefore, the sodium sulfite can help reduce the formation of colored bodies in the wash composition. However, sodium sulfite includes sodium, and sodium-containing compounds tend to produce efflorescent solids in the film. In some aspects, the sodium sulfite is present in the wash composition at a concentration of from about 0.05 to about 5 wt %. In some aspects, the sodium sulfite is present in the wash composition at a concentration of from about 0.05 to about 4 weight percent, or from about 0.05 to about 3 weight percent, or from about 0.05 to about 2 weight percent, all based on the total weight of the wash composition. In some aspects, the sodium sulfite is in an amount from about 0.5 to about 5 wt % of the detergent composition.

In some aspects, detergent compositions according to various aspects of the disclosure may include a performance polymer. In some aspects, the performance polymer is in an amount ranging from about 0.1 to about 10 wt % of the detergent composition. In some aspects, the performance polymer is in an amount ranging from about 1 to about 10 wt % of the detergent composition. In some aspects, the performance polymer is in an amount ranging from about 1 to about 5 wt % of the detergent composition. In some aspects, the performance polymer is one or more of a polyetheretherketone, a polyimide, a polyamide, a polymer composite, a polyethyleneimine, an ethoxylate, a polyethyleneimine ethoxylate, a propoxylate, or a polyethersulfone. In some aspects, combinations of at least two of a polyethyleneimine, an ethoxylate, a polyethyleneimine ethoxylate, and a propoxylate can be used as the performance polymer.

In some aspects, detergent compositions according to various aspects of the disclosure may include a fragrance and/or a dye. In some aspects, the fragrance and/or the dye are in an amount ranging from about 0.5 to about 2 wt % of the detergent composition. In some aspects, the fragrance and/or the dye are in an amount ranging from about 0.5 to about 5 wt % of the detergent composition. In some aspects, the fragrance and/or the dye are in an amount ranging from about 1 to about 2 wt % of the detergent composition. In some aspects, the dye is a blue dye, a yellow dye, a red dye, or a green dye. In some aspects, the dye can be color dyes commonly known in the art. In some aspects, the fragrance is a citrus scent, a floral scent, a clean scent, or a sweet scent. In some aspects, the fragrances can be neat oil fragrances, pro-fragrances, and encapsulated fragrances that are commonly known in the art.

In some aspects, detergent compositions according to various aspects of the disclosure may include a microbiocide, an algicide, and a fungicide. In some aspects, the microbiocide, algicide, and fungicide are in in an amount ranging from about 0.01 to about 5 wt % of the detergent composition. In some aspects, the microbiocide, algicide, and fungicide are in an amount ranging from about 0.01 to about 3 wt % of the detergent composition.

In some aspects, detergent compositions according to various aspects of the disclosure further comprises an alkanolamine, such as, for example monoethanolamine. In some instances, the monoethanolamine ranges in an amount from about 1 to about 10 wt % of the detergent composition.

In some aspects, detergent compositions according to various aspects of the disclosure may include a combination of two or more of, or all of, a defoamer, a buffer, an optical brightener, an anti-redeposition polymer, and a preservative. In some aspects, the combined amount of two or more of, or all of, the defoamer, the buffer, the optical brightener, the anti-redeposition polymer, and the preservative ranges from about 5 to about 15 wt % of the detergent composition. In some aspects, detergent compositions according to various aspects of the disclosure may include a combination of two or more of, or all of a defoamer, a buffer, an optical brightener, an anti-redeposition polymer, and a preservative. In some aspects, the combined amount of two or more of, or all of the defoamer, the buffer, the optical brightener, the anti-redeposition polymer, and the preservative ranges from about 5 to about 15 wt % of the detergent composition. In some aspects, the buffer is a sodium carbonate. In some aspects, the buffer is a sodium bicarbonate. In some aspects, the buffer is a carbonate or bicarbonate. In some aspects, an example of a suitable bicarbonate buffer is sodium bicarbonate. In some aspects, the buffer is a borate. In some aspects, the buffer is monoethanolamine. In some aspects, the buffer is lysine. In some aspects, the buffer is a sugar amine. In some aspects, an example of a suitable sugar amine buffer is glucosamine. In some aspects, the buffer is a citrate.

In some aspects, the detergent composition comprises a peptide in an amount ranging from about 0.01 to about 5 wt % of the detergent composition, about 1 to about 35 wt % of a non-ionic surfactant, about 1 to about 40 wt % of an anionic surfactant, about 5 to about 70 wt % of a non-aqueous solvent or non-aqueous co-solvent mixture, and water.

In some aspects, the detergent composition comprises a peptide in an amount ranging from about 0.01 to about 5 wt % of the detergent composition, about 1 to about 30 wt % of a non-ionic surfactant, about 1 to about 35 wt % of an anionic surfactant, about 15 to about 65 wt % of a non-aqueous solvent or non-aqueous co-solvent mixture, and water.

In some aspects, the detergent composition comprises a peptide in an amount ranging from about 0.01 to about 5 wt % of the detergent composition, about 1 to about 30 wt % of an alkoxylated alcohol, about 1 to about 35 wt % of a linear alkyl sulfonate, a linear alkylbenzene sulfonate, a sodium lauryl sulfate, a sodium lauryl ether sulfate, and any combination thereof, about 15 to about 65 wt % of a non-aqueous solvent or non-aqueous co-solvent mixture comprising glycerin, propylene glycol, polyethylene glycol, ethanol, and any combination thereof, and water.

In some aspects, the detergent composition consists of a peptide in an amount ranging from about 0.01 to about 5 wt % of the detergent composition, about 1 to about 30 wt % of a non-ionic surfactant, about 1 to about 35 wt % of an anionic surfactant, about 15 to about 65 wt % of a non-aqueous solvent or non-aqueous co-solvent mixture, water, and at least one ingredient selected from the group consisting of: a fatty acid, a strong base, one or more enzymes, a performance polymer, an alkanolamine, a fragrance, a dye, and any combination thereof.

In some aspects, the detergent composition consists essentially of a peptide, a non-ionic surfactant, an anionic surfactant, a non-aqueous solvent or non-aqueous co-solvent mixture, water, and at least one ingredient selected from the group consisting of: a fatty acid, a strong base, one or more enzymes, a performance polymer, an alkanolamine, a fragrance, a dye, and any combination thereof.

In some aspects, the detergent composition consists essentially of a peptide, a non-ionic surfactant, an anionic surfactant, a non-aqueous solvent or non-aqueous co-solvent mixture, and water.

Also provided herein are methods of cleaning laundry. Methods of cleaning laundry may include combining an amount of a detergent composition according to various aspects of the disclosure with an amount of water to form a diluted detergent composition having from about 0.1 grams to about 1 gram of the detergent composition per liter of water and cleaning the laundry using the diluted detergent composition. In some aspects the diluted detergent composition has a concentration of about 0.01 to about 10 grams, alternatively from about 0.02 to about 8 grams, alternatively from about 0.03 to about 6 grams, alternatively from about 0.04 to about 6 grams, alternatively from about 0.05 to about 5 grams, alternatively from about 0.06 to about 4 grams, alternatively from about 0.07 to about 3 grams, alternatively from about 0.08 to about 2 grams, and alternatively from about 0.09 to about 1.5 grams of the detergent composition per liter of water. In some aspects, the diluted detergent composition has a concentration of about 1 to about 2 grams of the detergent composition per liter of water.

### EXAMPLES

### Example 1—Peptides as Chlorine Scavengers in Laundry Detergents

The following formulations were batched using a standard overhead mixer and will be referred to “Formulas 1 and 2” as listed below in Table 1:

TABLE 1

| | (Control, | (Inventive, |
|---|---|---|
| Component (% activity) | Formula 1) wt % | Formula 2) wt % |
| Non-aqueous solvent | 51.78 | 51.28 |
| (Glycerin, PEG 400, 200 | | |
| Proof Ethanol) | | |
| Non-ionic surfactant | 23.074 | 23.074 |
| (Alcohol Ethoxylate) | | |
| Water | 10.5 | 10.5 |
| Coconut Fatty Acid and | 10.8 | 10.8 |
| Anionic Surfactant | | |
| (SLES, 3 EO (70%)) | | |
| Peptide (50% solution) | 0 | 0.5 |
| Bittering Agent (25%), | 3.845 | 3.845 |
| Sodium Sulfite (15%), | | |
| Chelant (34%), pH | | |
| Adjusters (50%) | | |

SLES: sodium lauryl ether sulfate
EO: ethoxylate

The target dose detergent per wash is approximately 18.8 grams and the estimated amount of water in High Efficiency Washing Machine is 31.4 L. Proportionally, 0.6 grams of detergent will go into 1 L of water (benchtop scale).

With a starting chlorine concentration of 1 ppm in the water, the following results were gained after adding 0.6 grams of Formula 1 and 2 to 1 Liter of 1 ppm chlorinated water: Formula 2 had L-Lysine, 50% substituted for the peptide as L-Lysine contains the functional amino acid of the peptide, and thus can show the effect.

TABLE 2

| Dose | | Start ppm | 15 seconds | 30 seconds | 1 minute | 2 minutes |
|---|---|---|---|---|---|---|
| 0.6 g in | Control | 1 | 1 | 1 | 0.5 | 0.5 |
| 1 L | (Formula 1) | | | | | |
| | Formula 2 | 1 | 0 | 0 | 0 | 0 |

Formulations 1+2 had a viscosity of about 150 cP at 25° C.

Enzymes and polymers known to the art, such as protease, mannanase and amylase (among others) can be added to formulations 1 and 2, to improve washing efficacy as well as performance polymers such as polyethylene imines or anti-redeposition polymers.

The formulations in Example 1 can be further enclosed within polyvinyl alcohol film, standard to the industry, to create unit dose or liquid detergent packs.

The improvement in chlorine scavenging efficacy can be observed with Formula 2, with Formula 2 scavenging all chlorine within 15 seconds vs. the Control (Formula 1) not scavenging any chlorine until 1 minute. Formula 1 was only capable of scavenging half of the chlorine as well.

### Example 2

Another unit dose example is as followed in Table 3:

TABLE 3

| Component (% activity) | Formula 3 (wt %) | Formula 4 (wt %) |
|---|---|---|
| Water | 7.03 | 7.03 |
| Non-aqueous solvent (glycerin, | QS to 100 | QS to 100 |
| propylene glycol, 200 proof | | |
| ethanol) | | |
| Anionic Surfactant (LAS and Fatty | 31.77 | 31.77 |
| Acid) | | |
| Nonionic Surfactant | 24.10 | 24.10 |
| (alcohol ethoxylate - C₁₃₋₁₅- 8 EO) | | |
| pH Adjuster, Optical Brightener, | 16.70 | 16.69 |
| Performance Polymer (80%), | | |
| Bittering Agent, Anti-Redeposition | | |
| Polymer (70%), Fragrance and Dye | | |
| Peptide (50%) | 0 | 0.5 |
| Enzyme | 0.22 | 0.22 |

LAS: linear alkyl sulfonate
EO: ethoxylate

Especially peptides which have domains that show affinity to bind to different types of textiles and do contain a high number of amino acids which are susceptible to oxidation (like i.e. in methionine, lysine, cysteine, and others) are most preferred. The most effective peptides are expected to be in the length range of 10-60 amino acids and do contain at least two, preferably more than two amino acids susceptible to oxidation. Furthermore, peptides according to various aspects of the disclosure can include one or more linking domains such as, for example, SEQ ID NO: 1-3, 7-12, 27, and 32.

### Example 3—Peptides as Chlorine Scavengers in Laundry Detergents

The following formulations were batched using a standard overhead mixer and will be referred to “Formula 1 and 2” as listed below in Table 4:

TABLE 4

| | Raw | Wt % of | Wt % of |
|---|---|---|---|
| | Material | Formula 1 | Formula 2 |
| Component | Activity | (Control) | (Inventive) |
| Water, demineralized | 100 | q/s to 100 | q/s to 100 |
| Citric Acid | 50 | 0.2 | 0.2 |
| Sodium tetraborate 5H₂O | 100 | 0.5 | 0.5 |
| 50% NaOH | 50 | 2.4 | 2.4 |
| Fatty alcohol ethoxylate | 100 | 16.2 | 16.2 |
| (non-ionic surfactant) | | | |
| Sodium Laureth Sulfate, | 29 | 5.5 | 5.5 |
| 3EO (anionic surfactant) | | | |
| Coconut Fatty Acid, | N/A | 5 | 5 |
| Anti-redeposition Polymer, | | | |
| Chelating agent, Preservative, | | | |
| Aqueous Enzyme Solution | | | |
| (Protease, Amylase, Mannanase | | | |
| blend), Propylene Glycol/ | | | |
| Ethanol | | | |
| Linear Alkyl Benzene | 96 | 9 | 9 |
| Sulfonic Acid | | | |
| Peptide | 50 | 0 | 0.5 |

EO: ethoxylate

The target dose of detergent per wash was 45 grams, and the estimated amount of water in a High Efficiency Washing Machine was 31.4 L. Proportionally, 1.43 grams of detergent was added into 1 L of water for benchtop scale.

With a starting chlorine concentration of 1 ppm of hypochlorite in the water, the results in Table 2 were observed after adding 1.43 grams of Formula 1, 2, or 3 to 1 L of the chlorinated water. Formula 2 had L-Lysine, 50% substituted for the peptide as L-Lysine contains the functional amino acid of the peptide, and thus can show the effect.

TABLE 5

| | | Initial | | | | |
|---|---|---|---|---|---|---|
| | | Chlorine | Chlorine Concentration after | | | |
| | Solution | Concentration | Contact with Formula (ppm) | | | |
| Formula | Agitated | (ppm) | 15 sec | 30 sec | 60 sec | 120 sec |
| 1 (control) | Yes | 1 | 0.5 | 0.5 | 0 | 0 |
| 2 (inventive) | Yes | 1 | 0 | 0 | 0 | 0 |

An exemplary enzymatic formulation containing peptides are described below in Table 6:

TABLE 6

| | Raw | | | | |
|---|---|---|---|---|---|
| | Material | | | | |
| | Activity | Formula 5 | Formula 6 | Formula 7 | Formula 8 |
| Water softened | 100.00% | q/s to 100 | q/s to 100 | q/s to 100 | q/s to 100 |
| Laureth-6 plant-based | 100.00% | 27.2 | 27.2 | 27.2 | 27.2 |
| EO, Anionic Surfactant | | | | | |
| and Fatty Acid | | | | | |
| Citric acid 50% solution | 50.00% | 4.5 | 4.5 | 4.5 | 4.5 |
| Sodium hydroxide 50% | 50.00% | Adjusted | Adjusted | Adjusted | Adjusted |
| (Membrane grade) | | to pH 8 | to pH 8 | to pH 8 | to pH 8 |
| Peptide | 50% | 0.25 | 0.5 | 0.75 | 1 |
| Polymer, Chelant, | N/A | 4.77 | 4.77 | 4.77 | 4.77 |
| Preservative, Aqueous | | | | | |
| Enzyme Solution | | | | | |
| (Protease, Amylase, | | | | | |
| Mannanase blend) | | | | | |
| (100%) | | | | | |

EO: ethoxylate

Further, a high pH, non-enzymatic formula example is as followed in Table 7:

TABLE 7

| | Raw Material | Formula 14 | Formula |
|---|---|---|---|
| Component | Activity | (standard) | 15 |
| Water | 100% | q/s to 100 | q/s to 100 |
| Nonionic surfactant | Approx. 88% as a | 17.14 | 17.14 |
| (fatty alcohol ethoxylate C₁₂₋₁₅ 7EO) and | blend | | |
| Anionic surfactant | | | |
| (sodium laureth sulfate, 3EO) | | | |
| Anionic surfactant/Defoamer | 100% | 10.95 | 10.95 |
| (coconut fatty acid derived from palm) and | | | |
| sodium carbonate and Optical Brightener and | | | |
| Anti-Redeposition Polymer and Preservative | | | |
| Chelating agent | 34% | 1.18 | 1.18 |
| (tetrasodium iminodisuccinate) | | | |
| Peptide | 50% | — | 0.15 |

EO: ethoxylate

Especially peptides which have domains that show affinity to bind to different types of textiles and do contain a high number of amino acids which are susceptible to oxidation (like i.e. Methionine, Lysine, Cysteine and others) are most preferred. The most effective peptides are expected to be in the length range of 10-60 amino acids and do contain at least two, preferably more than two amino acids susceptible to oxidation. Furthermore, peptides according to various aspects of the disclosure can include one or more linking domains such as, for example, SEQ ID NO: 1-3, 7-12, 27, and 32.

Table 8 lists various sequences used in the detergent compositions.

TABLE 8

| SEQ ID NO | Sequence |
|---|---|
| 1 | NGLLIPQFLVASGGGGSRSIVTFSLRQNRGGGGSNGLLIPQFLV |
| | AS |
| 2 | NGLLIPQFLVASGGGGSRALQALRALQALEALGGGGSNGLLIPQ |
| | FLVAS |
| 3 | NGLLIPQFLVASGGGGSRALRALQALEALEALGGGGSNGLLIPQ |
| | FLVAS |
| 4 | RSIVTFSLRQNR |
| 5 | RALQALRALQALEAL |
| 6 | RALRALQALEALEAL |
| 7 | NGLLIPQFLVASGGGGSMSDYQMDM |
| 8 | RALRALQALEALEALGGGGSMSDYQMDM |
| 9 | RALQALRALQALEALGGGGSMSDYQMDM |
| 10 | NGLLIPQFLVASGGGGSMDMQGRYMDR |
| 11 | RALRALQALEALEALGGGGSMDMQGRYMDR |
| 12 | RALQALRALQALEALGGGGSMDMQGRYMDR |
| 13 | GGGGS |
| 14 | GGGGSGGGGS |
| 15 | GGGGSGGGGSGGGGS |
| 16 | GGGGSGGGGSGGGGSGGGGGGS |
| 17 | GGGGGGGG |
| 18 | GGGGGG |
| 19 | EAAAK |
| 20 | EAAAKEAAAK |
| 21 | EAAAKEAAAKEAAAK |
| 22 | AEAAAKEAAAKEAAAKEAAAKALEAAAAAKEAAAKEAAAKEAAA |
| | KA |
| 23 | AEAAAKEAAAKA |
| 24 | PAPAP |
| 25 | SGGG |
| 26 | GGGS |
| 27 | MYMDMKDVMMKYDMAMMEMKMDSGGGRSIVTFSLRQNRGGGSMY |
| (P8-Scavenger-1) | MDMKDVMMKYDMAMMEMKMD |
| 28 | MYMDMKDVMMKYDMAMMEMKMDRSIVTFSLRQNRMYMDMKDVMM |
| (P8-Scavenger-2) | KYDMAMMEMKMD |
| 29 | MKMYMKMDRSIVTFSLRQNRMKMYMKMD |
| (P8-Scavenger-3) | |
| 30 | MMYMMYMRSIVTFSLRQNRMMYMMYM |
| (P8-Scavenger-4) | |
| 32 | KYGDMKDVKMKYDKACSEMKYDSGGGRSIVTFSLRQNRGGGSKY |
| (P8-Scavenger-5) | GDMKDVKMKYDKACSEMKYD |
| 33 | KYGDMKDVKMKYDKACSEMKYDRSIVTFSLRQNRKYGDMKDVKM |
| (P8-Scavenger-6) | KYDKACSEMKYD |
| 34 | MYMDMKDVMMKYDMAMMEMKMD |
| 35 | MKMYMKMD |
| 36 | MMYMMYM |
| 37 | NGLLIPQFLVAS |
| 38 | KYGDMKDVKMKYDKACSEMKYD |

### Example 4. Small Scale E Valuation of Chlorine Scavenging Rinse Carry-Over

The following describes a small scale method for evaluating the efficacy of peptide reagents for binding to fabric and scavenging free chlorine in a simulated wash and rinse cycle.

A 400 mL glass beaker equipped with a cross-shaped stir bar is used as the washing/rinsing vessel. Two hundred grams of prepared water is weighed into the beaker. The water is prepared with 120 ppm hardness (calcium and magnesium, 3:1 ratio), and 3 ppm free chlorine via a bleach solution. The free chlorine level is monitored using an Insta-Test Analytic free chlorine test strip from LaMotte. The wash water is heated on a hot plate to 90° F. (32.2° C.).

The chemistry of interest is injected into the wash water via a micropipette. For the reported tests, a 1% by weight solution of selected peptide in deionized water is prepared and injected into the wash water to deliver 20 mg of active ingredient (2 mL). For P8-Scavenger 4 (SEQ ID NO: 30), a 0.5% by weight solution was prepared in a 1:1 Water:Ethanol mixture and dosed appropriately. The peptide solution is stirred for 1 minute.

Pre-cut cotton/polyester fabric sheets (55/45 ratio from SDL Atlas) weighed out to 4 grams are added directly to the wash water. The water, peptide, and ballast mixture are allowed to stir for 12 minutes. Afterwards, the ballast is removed from the water and placed in a device which applies centrifugal force to remove excess water from the sample. The ballast is spun out for about 2 minutes.

Separately, a new 400 mL beaker is prepared with 200 grams of water (120 ppm hardness, 3 ppm free chlorine) to simulate the rinse cycle. The temperature is set to 60° F. (about 15.5° C.). The free chlorine level is checked with a test strip. The treated ballast is added to the rinse water and stirred for 4 minutes. The chlorine scavenging rate is monitored by using the free chlorine test strips at various time points (e.g. 2 minutes, 4 minutes).

The procedure was conducted using polylysine (>95% purity) from MarkNature as a control, as well as the specialized peptide constructs. Table 4. below shows the free chlorine level (FCL) in the simulated rinse water over the course of 4 minutes, as determined based on the visual indicator test strips.

TABLE 9

| | | | FCL in | FCL in | | FCL in | | FCL in | |
|---|---|---|---|---|---|---|---|---|---|
| | Amino | FCL in | Rinse | Rinse | | Rinse | | Rinse | |
| Sample | Acid | Rinse | (1 | (2 min- | | (3 | | (4 min- | |
| Identify | Sequence | (Initial) | minute) | utes) | | minutes) | | utes) | |
| Poly-lysine | — | 3.0 ppm | 2.0 ppm | 1.0 | ppm | 0.5 | ppm | 0.25 | ppm |
| P8-SRP (SEQ | NGLLIPQFL | 3.0 ppm | 2.5 ppm | 2.0 | ppm | 1.5 | ppm | 1.0 | ppm |
| ID NO: 1) | VASGGGGSR | | | | | | | | |
| | SIVTFSLRQ | | | | | | | | |
| | NRGGGGSNG | | | | | | | | |
| | LLIPQFLVA | | | | | | | | |
| | S | | | | | | | | |
| P8- | MKMYMKMDR | 3.0 ppm | 1.5 ppm | 0.25 | ppm | 0 | ppm | 0 | ppm |
| Scavenger-3 | SIVTFSLRQ | | | | | | | | |
| (SEQ ID NO: | NRMKMYMKM | | | | | | | | |
| 29) | D | | | | | | | | |
| P8- | MMYMMYMRS | 3.0 ppm | 2.0 ppm | 1.0 | ppm | 0.5 | ppm | 0.25 | ppm |
| Scavenger-4 | IVTFSLRQN | | | | | | | | |
| (SEQ ID NO: | RMMYMMYM | | | | | | | | |
| 30) | | | | | | | | | |

It was observed that P8-Scavenger 3 (SEQ ID NO: 29) was highly effective for scavenging free chlorine in the simulated rinse water compared to polylysine, indicating effective carry-over on the ballast fabric. P8-Scavenger-4 (SEQ ID NO: 30) provided similar chlorine scavenging efficacy to polylysine, whereas P8-SRP (SEQ ID NO: 1) only provided modest scavenging of free chlorine in the rinse water. Therefore, the peptide sequence has a significant impact on its scavenging efficacy in simulated rinse water. The breadth and scope of the present invention should not be limited by any of the above-described exemplary embodiments, but should be defined only in accordance with the following claims and their equivalents.

What is claimed is:

1. A peptide comprising:
(i) one or more textile binding domains, wherein the one or more textile binding domains comprise an amino acid sequence having at least 70%, at least 75%, at least 80%, at least 85%, at least 90%, at least 95%, at least 97%, at least 99%, or 100% identity to SEQ ID NO: 4; and
(ii) one or more domains susceptible to oxidation, wherein the one or more domains susceptible to oxidation comprise an amino acid sequence having at least 70%, at least 75%, at least 80%, at least 85%, at least 90%, at least 95%, at least 97%, at least 99%, or 100% identity to any of SEQ ID NOs: 34-38.

2. The peptide of claim 1, comprising one textile binding domain and two domains susceptible to oxidation.

3. The peptide of claim 1, further comprises one or more linking domains.

4. The peptide of claim 3, comprising one textile binding domain, two domains susceptible to oxidation, and two linking domains.

5. The peptide of claim 1, wherein the peptide comprises an amino acid sequence having at least 70%, at least 75%, at least 80%, at least 85%, at least 90%, at least 95%, at least 97%, at least 99%, or 100% identity to any of SEQ ID NOs: 1-3, 5-12, 27-30, and 32-33.

6. A peptide comprising:
(i) one or more textile binding domains; and
(ii) one or more domains susceptible to oxidation, wherein the domains susceptible to oxidation are selected from:
(1) a domain comprising about 45% of the amino acids of the one or more domains susceptible to oxidation are M and about 55% of the amino acids are selected from Y, D, K, V, A, and E;
(2) a domain comprising about 50% of the amino acids of the one or more domains susceptible to oxidation are M and about 50% of the amino acids are selected from K, Y, and D;
(3) a domain comprising about 70% of the amino acids of the one or more domains susceptible to oxidation are M and about 30% of the amino acids are Y;
(4) a domain comprising about 25% of the amino acids of the one or more domains susceptible to oxidation are L and about 75% of the amino acids are N, G, I, P, Q, F, V, A, and S; and
(5) a domain comprising about 40% of the amino acids of the one or more domains susceptible to oxidation are selected from M and K and about 60% of the amino acids are selected from Y, G, D, V, A, C, S, and E.

7. The peptide of claim 6, wherein the one or more domains susceptible to oxidation comprise an amino acid sequence having at least 70%, at least 75%, at least 80%, at least 85%, at least 90%, at least 95%, at least 97%, at least 99%, or 100% identity to any of SEQ ID NOs: 34-38.

8. The peptide of claim 6, wherein the one or more textile binding domain comprises an amino acid sequence having at least 70%, at least 75%, at least 80%, at least 85%, at least 90%, at least 95%, at least 97%, at least 99%, or 100% identity to SEQ ID NO: 4.

9. A peptide comprising an amino acid sequence having at least 70%, at least 75%, at least 80%, at least 85%, at least 90%, at least 95%, at least 97%, at least 99%, or 100% identity to any of SEQ ID NOs: 1-3, 5-12, 27-30, and 32-33.

10. A detergent composition comprising the peptide of claim 1.

11. The detergent composition of claim 10, further comprising:
(a) about 1 to about 30 wt % of a non-ionic surfactant;
(b) about 1 to about 35 wt % of an anionic surfactant;
(c) about 15 to about 65 wt % of a non-aqueous solvent or non-aqueous co-solvent mixture; and
(d) water,
wherein the peptide is present in an amount ranging from about 0.01 to about 5 wt % of the detergent composition.

12. The detergent composition of claim 11, wherein the non-ionic surfactant comprises an alkoxylated alcohol.

13. The detergent composition of claim 11, wherein the anionic surfactant comprises a linear alkyl sulfonate, a linear alkylbenzene sulfonate, a sodium lauryl sulfate, a sodium lauryl ether sulfate, and any combination thereof.

14. The detergent composition of claim 11, wherein the non-aqueous solvent or non-aqueous co-solvent mixture comprises glycerin, propylene glycol, polyethylene glycol, ethanol, and any combination thereof.

15. The detergent composition of claim 11, further comprising at least one ingredient selected from the group consisting of: a fatty acid, a strong base, one or more enzymes, a performance polymer, an alkanolamine, a fragrance, a dye, and any combination thereof.

16. The detergent composition of claim 15, wherein the detergent composition comprises one or more enzymes.

17. The detergent composition of claim 16, wherein the detergent composition has a pH of about 7 to about 9.

18. The detergent composition of claim 11, further comprising a pouch encapsulating the detergent composition, wherein the pouch is made of a water-soluble material.

19. A method of cleaning textiles, the method comprising:
combining the detergent composition of claim 11 with water to form a diluted detergent composition having from about 1 to about 2 grams of the detergent composition per liter of water; and
cleaning the textiles using the diluted detergent composition.

20. The method of claim 19, wherein the textiles are made of natural materials, synthetic materials or blends thereof.
